(19) United States
(12) Patent Application Publication
(10) Pub. No.: US 2018/0106782 A1
(43) Pub. Date: Apr. 19, 2018

(54) HIGH THROUGHPUT CARDIOTOXICITY SCREENING PLATFORM

(71) Applicant: The Board of Trustees of the Leland Stanford Junior University, Stanford, CA (US)

(72) Inventors: Beth L. Pruitt, Stanford, CA (US); Alexandre Ribeiro, Stanford, CA (US); Robin Wilson, Stanford, CA (US)

(21) Appl. No.: 15/784,620

(22) Filed: Oct. 16, 2017

Related U.S. Application Data
(60) Provisional application No. 62/409,284, filed on Oct. 17, 2016.

(51) Int. Cl. G01N 33/50; G01N 33/487; G01N 33/483; C09K 11/06; C12N 5/00; A61K 35/34; A61K 49/00

(52) U.S. Cl. CPC G01N 33/5061; G01N 33/48721; G01N 33/48728; G01N 33/4833; C09K 11/06; G01N 2203/0089; G01N 33/5029; G01N 33/5073; A61K 35/34; A61K 49/0073; C12N 5/0068

(57) ABSTRACT

Systems for assaying human induced pluripotent stem cell-derived cardiomyocytes (hiPSC-CMs) are provided. Aspects of the systems include a traction force microscopy substrate, such as a traction force microscopy hydrogel (TFM-hydrogel), having an adhesion protein domain on a surface thereof; a video imager configured to obtain video data from an hiPSC-CM present on the adhesion protein domain; and a processing module configured to receive the video data and derive a parameter of the hiPSC-CM therefrom. Also provided are methods of using the systems.

### CROSS-REFERENCE TO RELATED APPLICATIONS

Pursuant to 35 U.S.C. § 119(e), this application claims priority to the filing date of U.S. Provisional Patent Application Ser. No. 62/409,284 filed on Oct. 17, 2016; the disclosure of which application is herein incorporated by reference.

### GOVERNMENT RIGHTS

This invention was made with Government support under contract MIKS-1136790 awarded by the National Science Foundation. The Government has certain rights in the invention.

### INTRODUCTION

Cardiomyocytes (CMs) are the muscle cells of the myocardium that collectively generate the mechanical output required for heart function. (Brady, 1991) The mechanical output of CMs originates from the intracellular contractile activity of sarcomeres aligned in series along myofibrils. (Nadal Ginard, et al., 1989) Human induced pluripotent stem cells (hiPSCs) can be differentiated towards beating CMs (hiPSC-CMs).(Talkhabi et al., 2016) However, myofibrils in hiPSC-CMs are disarrayed in opposition to the well-organized myofibrils in primary CMs. (Yang et al., 2014)

Until very recently, the disarray of myofibrils in hiPSC-CMs was a limiting factor for calculating the mechanical output of these cells and assay cardiac function in vitro. (Yang et al., 2014) However, micropatterning (ppatterning) of hiPSC-CMs on substrates can induce the intracellular alignment of myofibrils (Wang et al., 2014) and therefore enhance the maturity of their contractile activity. (Ribeiro et al., 2015a; Ribeiro et al., 2015b) By ppatterning hiPSC-CMs on compliant substrates of known mechanical properties, one can calculate their mechanical output through non-destructive and minimally invasive microscopy-based approaches. (Ribeiro et al., 2015a; Ribeiro et al., 2015b; Kijlstra et al., 2015)) These approaches include traction force microscopy to calculate cell-generated tractions, analyzing the contractile movement of hiPSC-CMs, measuring the displacement of myofibrils and the varying length of sarcomeres.

However, these analytical strategies have been often developed independently of one another, differ from lab to lab and are not easily available to researchers in need of performing these studies.

### SUMMARY

Systems for assaying human induced pluripotent stem cell-derived cardiomyocytes (hiPSC-CMs) are provided. Aspects of the systems include a traction force microscopy substrate, such as traction force microscopy hydrogel (TFM-hydrogel), having an adhesion protein domain on a surface thereof; a video imager configured to obtain video data from an hiPSC-CM present on the adhesion protein domain; and a processing module configured to receive the video data and derive a parameter of the hiPSC-CM therefrom. Also provided are methods of using the systems.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1, panels A-L. Registering the contractile mechanical output of μpatterned hiPSC-CMs. A, Three classes of videos of beating μpatterned hiPSC-CMs were acquired with microscopy: brightfield videos, videos of microbeads embedded in the deformable gel substrate and videos of moving myofibrils. B, A region of interest (ROI) was defined around the contour of the cell and the movement within this region was analyzed with cross-correlation from brightfield videos. C, Cell average displacement (d) due to the contractile activity of beating within the ROI was quantified and plotted as a function of time: d-curve. D, Average velocity of displacement (V) within the ROI was calculated from the first derivative of displacement and plotted as a function of time: V-curve. E, Displacement of microbeads embedded in the gel substrate is quantified with cross-correlation from fluorescent videos. An ellipse calculated from the dimension of the ROI is automatically drawn to limit the calculation of displacement to this region. F, d-curve of microbeads was plotted as a function of time. G, V-curve of microbeads is plotted as a function of time. H, Contractile force (ΣF) was estimated with traction force microscopy from d of microbeads and plotted as a function of time: F-curve. I, Power (P) was calculated by multiplying ΣF by V of microbeads and plotted as a function of time: P-curve. J, The regions occupied by sarcomeres within labeled myofibrils were skeletonized. K, d-curve of myofibrils and L, myofibril V-curve.

FIG. 2. Cross-correlation methods to quantify movement from videos.

We obtained d-curves within a beating μpatterned hiPSC-CM from a bright field video (Online Movie IV) by using three different cross-correlation approaches: Ncorr⁹, PIVIab¹⁰ and ImageJ PIV.¹¹ These analyses were repeated for the same video after decreasing the image resolution of its frames and adding image noise.

FIG. 3, panels A-C. Kinetic properties of contractile cycles. A, We determined three different kinetic parameters from V-curves that represent each contractile cycle: V_{C} is the peak velocity of contraction, V_{R} is the peak velocity of relaxation and \hat{t} is the time between the peak velocity of contraction and the peak velocity of relaxation. The presented V-curve was calculated from the displacement of microbeads. B, Variations in the d-curve derived from videos of moving microbeads was analyzed after slowly increasing the concentration of caffeine in the extracellular milieu. We determined maximum values (dashed lines) and minimum values (circles) of d. C, P was also analyzed while increasing the concentration of caffeine. P was calculated by multiplying F by V to determine peak P of contraction (P_{C}), peak P of relaxation (P_{R}) and \hat{t}. Peaks of P are marked with dashed lines.

FIG. 4, panels A-I. Single-cell analysis of isoproterenol (ISO)-induced variations in mechanical output. We added isoproterenol to the extracellular milieu of a beating μpatterned hiPSC-CM at two different concentrations: 0.1 μM and 1 μM. A, Heat map of cell-generated traction stresses on the surface of the gel substrate were estimated with traction force microscopy and F was calculated within the region delimited by an ellipse around the cell. B, Myofibrils were fluorescently labeled in the analyzed μpatterned hiPSC-CM (Online Movie V) and imaged for quantification of myofibril movement. C, We also acquired a brightfield video of the analyzed single cell. D, F-curves and E, P-curves were estimated from videos of moving microbeads acquired before and after the cell being exposed to different concentrations of isoproterenol. F, and G, respectively present d-curves and V-curves calculated from videos of moving myofibrils before and after adding isoproterenol. H, and I, respectively show d-curves and V-curves obtained from bright field videos of the cell at different isoproterenol concentrations.

FIG. 5, panels A-K. Variation of parameters of mechanical output induced by isoproterenol (ISO). Parameters of mechanical output were determined from videos of moving microbeads with traction force microscopy for 6 different μpatterned hiPSC-CMs at two different concentrations of isoproterenol: 0.1 μM and 1 μM. We then calculated variation of each parameter relative to its value when isoproterenol is absent from the extracellular milieu. A, Representative F-curve estimated for a μpatterned hiPSC-CM before adding isoproterenol. B, F-curve after exposing the cell to 0.1 μM of isoproterenol. C, F-curve after adding isoproterenol to achieve a concentration of 1 μM of isoproterenol. D-K, Variation of parameters of mechanical output. D, Variation of dₘₐₓ, E, Variation of V_{C}. F, Variation of V_{R}. G, Variation of L H, Variation of f. I, Variation of F. J, Variation of P_{C}. K, Variation of P_{R}. *P<0.05, **P<0.01 and ***P<0.005 by unpaired Wilcoxon-Mann-Whitney rank-sum test. Error bars represent the standard error of the mean; n.s., not significant.

FIG. 6, panels A-I. Single-cell changes in mechanical output induced by omecamtiv mecarbil (OM). Omecamtiv mecarbil was added to the extracellular milieu of a beating μpatterned hiPSC-CM at a concentration of 0.1 μM and we acquired videos of microbeads in the substrate, of moving myofibrils and of the cell before and after adding omecamtiv mecarbil to analyze changes in mechanical output. A, Fluorescently labeled myofibrils before adding omecamtiv mecarbil (Online Movie VIII). B, Accute tightening of sarcomeres detected within 10 seconds after adding omecamtiv mecarbil (Online Movie IX) C, Chronic damage of myofibrils imaged 2 minutes after adding omecamtiv mecarbil (Online Movie X). D, F-curves and E, P-curves were estimated from videos of moving microbeads acquired before adding omecamtiv mecarbil and after acute and chronic exposure. F, and G, respectively present d-curves and V-curves calculated from videos of moving myofibrils. H, and I, respectively show d-curves and V-curves obtained from brightfield videos of the cell.

FIG. 7, panels A-H. Changes in sarcomere length (sl) and sarcomere shortening (ss) induced by isoproterenol (ISO) (A-D) and omecamtiv mecarbil (OM) (E-H). We measured sl and ss from the videos of myofibrils labeled in the beating μpatterned hiPSC-CMs presented in FIG. 4 and FIG. 6. A and E, Box plot of all average sl values calculated for all frames (n) of the analyzed videos. n=53 for the videos of the cell exposed to isoproterenol (Online Movies V, VI and VII) and n=50 for the videos of the cell exposed to omecamtiv mecarbil (Online Movies VIII, IX and X). B and F, detected maximum values of sl. C and G, minimum values of sl. D and H, ss calculated by subtracting minimum values of sl from maximum values of sl. Each point represents a value identified in the contractile curve of moving sarcomeres. *P<0.05, **P<0.01 by and ***P<0.005 by unpaired Wilcoxon-Mann-Whitney rank-sum test and by Bonferroni's all pairs comparison test; n.s., not significant with any test.

FIG. 8, panels A-G. Spatial (a_{θ}) and temporal (a_{δ}) asynchronicity parameters in μpatterned hiPSC-CMs with decreased MYBPC3 expression. The parameter a_{δ} is calculated from the offset times (δ) of intracellular displacement. (Methods Section) A, δ is determined for each pixel i within an ROI delimited by the borders of the cell by subtracting the time of each displacement peak for each pixel i by time of displacement peak for the average of displacement in the ROI. B, Representative ROI in a brightfield video of a beating μpatterned hiPSC-CM. C, Heat map of δ within the different pixels of the ROI. D-G, parameters calculated from analyzing μpatterned hiPSC-CMs that were TALEN-engineered to remove both copies of the MYBPC3 gene (−/−) and to remove one copy of the MYBPC3 gene (MYBPC3/−). MYBPC3/MYBPC3 cells were not TALEN-engineered. D, a_{θ}. E, a_{δ}. F, \hat{t}. *P<0.05, **P<0.01 and ***P<0.005 by unpaired Wilcoxon-Mann-Whitney rank-sum test and by Bonferroni's all pairs comparison test; n.s., not significant with any test.

FIG. 9, panels A-D. Variation of brightfield parameters of mechanical output induced by isoproterenol (ISO). The contractile displacement was analyzed with cross-correlation within a region of interest (ROI) delimited by the contour of the area of adhesion of 6 single beating hiPSC-CMs (FIG. 1, panel B) before and after adding isoproterenol at concentrations of 0.1 M and 1M. We calculated the isoproterenol-induced variation of parameters for each single cell. A, Variation in the average contractile displacement within the ROI. B, Variation in the average peak contraction velocity for each contractile cycle within the ROI. C, Variation in the average peak relaxation velocity for each contractile cycle within the ROI. D, Variation in the time between the peak velocity of contraction and the peak velocity of relaxation. Error bars represent the standard error of the mean; n.s., not significant.

FIG. 10, panels A-J. Variation of parameters of mechanical output induced by omecamtiv mecarbil (OM). We estimated parameters of mechanical output from traction force microscopy analysis to videos of microbeads moving due to tractions generated by 6 contractile μpatterned hiPSC-CMs. We calculated the variation in the values of these parameters after adding omecamtiv mecarbil at a concentration of 0.1 μM or 10 nM. A, Variation in the maximal displacement of microbeads. B, Variation in the peak velocity of contraction. C, Variation in the peak velocity of relaxation. D, Variation in the time between peak velocity of contraction and peak velocity of relaxation. E, Variation in beating rate. F, Variation in maximal force output. G, Variation in peak power of contraction. H, Variation in peak power of relaxation. *P<0.01 by unpaired Wilcoxon-Mann-Whitney rank-sum test. Error bars represent the standard error of the mean; n.s., not significant. I, Representative chronic (5 mins) change in the F-curve of a beating μpatterned hiPSC-CM after adding omecamtiv mecarbil at a concentration of 10 nM. J, Change in the F-curve of a beating μpatterned hiPSC-CM detected within 10 seconds of adding 0.1 μM of omecamtiv mecarbil.

FIG. 11, panels A-C. Detection of sarcomere damages in μpatterned hiPSC-CMs after adding omecamtiv mecarbil at different concentrations. We observed damaged myofibrils (green arrows) after adding omecamtiv mecarbil at A, 1 μM, B, 0.1 μM or C, 10 nM.

FIG. 12, panels A-D. Testing different strategies to calculate sarcomere length (sl) from an image of fluorescently labeled myofibrils in a single μpatterned hiPSC-CM. Sarcomeres were skeletonized for the image of labeled myofibrils for strategies A, B and C. A, The dominant sarcomere size was calculated from the two-dimensional spatial frequency plot that results from the Fourier transform of the skeletonized image. B, Average sarcomere length was determined from measuring the length between Z-lines in the image of skeletonized sarcomeres. We obtained heat maps representing the distribution of sl. C, Watersherd segmentation was used to isolate the space between Z-lines and we calculated sl from the main axis of the region occupied by a sarcomere. D, Lines were randomly drawn along myofibrils and we calculated sl from the intensity profile of these different lines.

FIG. 13, panels A-D. Detailed calculation of sarcomere length (sl) from videos of beating μpatterned hiPSC-CMs. A, Beating μpatterned hiPSC-CM with fluorescently labeled myofibrils (Online Movie XI). B, Skeletonization of sarcomeres (Online Movie XII) for each frame of a video of a beating μpatterned hiPSC-CM with labeled myofibrils. C, Heat map representing the distribution of sl within a μpatterned hiPSC-CM calculated for a frame of an acquired video (Online Movie XIII). D, Average values of sl calculated for each frame were plotted as a function of time and we selected maximal average sizes (red dots) and minimum average sizes (green squares) from these curves to calculate ss.

FIG. 14, panels A-D. Traction force microscopy approaches to estimate forces generated by μpatterned hiPSC-CMs. A, The cell borders defined a region of interested (ROI). Scale bar: 15 μm. B, Map of displacement of microbeads in the substrate was quantified with the cross-correlation algorithm Ncorr.{Blaber, 2015 #7} C, Unconstrained traction force microscopy estimates tractions (σ) directly from the displacement of microbeads.{Dembo, 1996 #16}{Landau, 1986 #20} We derived force only from the tractions in the space enclosed within the ellipse in green, which was calculated from the dimensions of the ROI (Materials and Methods). D, Constrained traction force microscopy estimates force generated within the ROI through an approach that initially considers the results from the unconstrained analysis.{Butler, 2002 #22} After using this approach, we observed tractions in regions that do not coincide with cell-generated deformations on the substrate (white arrows).

FIG. 15, panels A-B. Automated calculation of sarcomere length (sl) from the distance between Z-lines considering myofibril alignment. This method results in the approach presented in FIG. 12, panel B and FIG. 13 and was used for calculating sl and sarcomere shortening (ss). A, Illustration depicting how sl is calculated. A line is drawn between pairs of Z-lines in a frame that was skeletonized. This drawing procedure starts on the Z-line and ends on the Z-line to its right. The drawing considers the orientation of the myofibril going through each pair of Z-lines. B, Description on how the line between Z-lines is drawn. Considering the current point as the leading pixel of the line being drawn, there are 4 options for continuing drawing the line: top pixel (t), top-right pixel (tr), right pixel (r), bottom-right pixel (br) and bottom pixel (b). The decision of the pixel to extend the line is done based on the history of local myofibril orientation around the previous pixels of the line. The resulting line should also align along the average orientation the myofibril between the pair of Z-lines being processed. Otherwise, another decision will be made to meet these orientation criteria.

### DEFINITIONS

The term “induced pluripotent stem cell” (or “iPS cell”, or “iPSC”), as used herein, refers to a stem cell induced from a somatic cell, e.g., a differentiated somatic cell, and that has a higher potency than said somatic cell. iPS cells are capable of self-renewal and differentiation into mature cells, e.g. cells of mesodermal lineage or cardiomyocytes. iPS cells may also be capable of differentiation into cardiac progenitor cells.

As used herein, the term “stem cell” refers to an undifferentiated cell that can be induced to proliferate. The stem cell is capable of self-maintenance, meaning that with each cell division, one daughter cell will also be a stem cell. Stem cells can be obtained from embryonic, fetal, post-natal, juvenile or adult tissue. The term “progenitor cell”, as used herein, refers to an undifferentiated cell derived from a stem cell, and is not itself a stem cell. Some progenitor cells can produce progeny that are capable of differentiating into more than one cell type.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. In some embodiments, the individual is a human. In some embodiments, the individual is a murine.

### DETAILED DESCRIPTION

Systems for assaying human induced pluripotent stem cell-derived cardiomyocytes (hiPSC-CMs) are provided. Aspects of the systems include a traction force microscopy substrate, such as a traction force microscopy hydrogel (TFM-hydrogel), having an adhesion protein domain on a surface thereof; a video imager configured to obtain video data from an hiPSC-CM present on the adhesion protein domain; and a processing module configured to receive the video data and derive a parameter of the hiPSC-CM therefrom. Also provided are methods of using the systems.

Before the present methods and compositions are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Systems

As summarized above, systems for assaying human induced pluripotent stem cell-derived cardiomyocytes (hiPSC-CMs) are provided. Cardiomyocytes (CMs) are muscle cells that comprise cardiac muscle and generate the mechanical output necessary for heart function. CMs are physiologically characterized by intracellular contractile activity generated by sarcomeres aligned in series along myofibrils. Cardiomyocytes can have certain morphological characteristics. They can be spindle, round, triangular or multi-angular shaped, and they may show striations characteristic of sarcomeric structures detectable by immunostaining. They may form flattened sheets of cells, or aggregates that stay attached to the substrate or float in suspension, showing typical sarcomeres and atrial granules when examined by electron microscopy. Cardiomyocytes and cardiomyocyte precursors generally express one or more cardiomyocyte-specific markers. Cardiomyocyte-specific markers include, but are not limited to, cardiac troponin I (cTnI), cardiac troponin-C, cardiac troponin T (cTnT), tropomyosin, caveolin-3, myosin heavy chain (MHC), myosin light chain-2a, myosin light chain-2v, ryanodine receptor, sarcomeric a-actinin, Nkx2.5, connexin 43, and atrial natriuretic factor (ANF). Cardiomyocytes can also exhibit sarcomeric structures. Cardiomyocytes exhibit increased expression of cardiomyocyte-specific genes ACTC1 (cardiac a-actin), ACTN2 (actinin a2), MYH6 (a-myosin heavy chain), RYR2 (ryanodine receptor 2), MYL2 (myosin regulatory light chain, ventricular isoform, MYL7 (myosin regulatory light chain, atrial isoform), TNNT2 (troponin T type 2, cardiac), and NPPA (natriuretic peptide precursor type A), PLN (phospholamban). In some cases, cardiomyocytes can express cTnI, cTnT, Nkx2.5; and can also express at least 3, 4, 5, or more than 5, of the following: ANF, MHC, titin, tropomyosin, a-sarcomeric actinin, desmin, GATA-4, MEF-2A, MEF-2B, MEF-2C, MEF-2D, N-cadherin, connexin-43, β-1-adrenoreceptor, creatine kinase MB, myoglobin, a-cardiac actin, early growth response-I, and cyclin D2. As indicated above, one type of CM that may be assayed with systems described herein is human induced pluripotent stem cell-derived cardiomyocytes (hiPSC-CMs). hiPSC-CMs are pluripotent stem cells differentiated towards cardiomyocytes. Human-induced pluripotent stem cells (hiPSCs) are pluripotent stem cells generated from adult human tissue.

Aspects of the systems include a traction force microscopy substrate, such as a traction force microscopy hydrogel (TFM-hydrogel), having an adhesion protein domain on a surface thereof. Traction force microscopy substrate (TFM substrate) that may be employed in embodiments of the invention include polymeric structures having well-characterized mechanical behavior, where the polymeric structures are capable of sustaining cellular viability and have movement markers associate therewith. TFM substrates of interest may vary in material rigidity, and in some instances have a material rigidity ranging from 2 kPa to 100 kPa, such as 4 kPa to 50 kPa. Movement markers associated with the substrate may vary, where movement markers that may be associated include detectable particles, such as fluorescent beads, detectable proteins, such as fluorescently conjugated proteins, etc. Examples of TFM substrates that may be employed in embodiments of the invention include, but are not limited to, those TFM substrates described in: published United States Patent Application Publication Nos. 20170199175, 20170176415, 20140336072, 20140024045, 20140024041 and 20110189719, as well as published PCT Publication Nos. WO/2010/011407 and WO/2013/074972, the disclosures of which are herein incorporated by reference.

In some instances, the TFM substrate is a traction force microscopy hydrogel (TFM hydrogel). TFM-hydrogels of interest include optically transparent, colloidal polymer gels with well-characterized mechanical behaviors that are capable of sustaining cellular viability. TFM-hydrogels employed systems of the invention may include one or more synthetic or natural polymers, where polymers of interest include, but are not limited to: acrylamide, bisacrylamide, or dimethylsiloxane. In some instances, the polymers making up the hydrogel may be cross-linked. The water content of the hydrogels may also vary, and in some instances ranges from 75% to 95%. The hydrogels may also vary in material rigidity, and in some instances ranges from 4 kPa to 50 kPa, such as 2.3 kPa, 4.1 kPa, 8.6 kPa, 10 kPa, 16.3 kPa, and 30 kPa. In some instances, the TFM-hydrogels include fluorescent microbeads. Fluorescent microbeads of interest include polymeric beads that incorporate a fluorescent dye for use in monitoring movement via substrate displacement. The concentration of fluorescent microbeads in the hydrogel may vary, and is some instances is a concentration sufficient to calculate the forces generated by cells attached to the hydrogel surface. In some instances, the concentration for fluorescent microbeads ranges from 1×10⁸ microbeads/mL to 1×10¹⁰ microbeads/mL, such as 6.25×10⁹ microbeads/mL.

As indicated above, the TFM substrate includes an adhesion protein domain on a surface thereof. By “adhesion protein domain” is meant a domain or region, e.g., area, on a surface of the TFM-hydrogel that includes one or more adhesion proteins. In some instances, the adhesion protein domain covers the entirety of the surface. In some instances, the adhesion protein domain includes a plurality of distinct adhesion proteins of differing amino acid sequence. While the number of distinct adhesion proteins present in a given adhesion protein domain may vary, in some instances the number ranges from two to ten, such as two to five, e.g., two to four. Any convenient adhesion protein(s) may be present in an adhesion protein domain. Specific adhesion proteins of interest include, but are not limited to: fibronectin, collagen I, collagen IV, laminin, vitronectin and the like. In some instances, a given adhesion protein domain includes a mixture of a number of different adhesion proteins, where such mixtures may vary, and include matrigel, etc. Adhesions proteins may be employed that provide for simple release of cells following imaging in the system, e.g., for further analysis of analysis. For example, adhesions proteins in the adhesion protein domains may include stimulus labile moieties, e.g., enzyme cleavage sites, chemical cleavage sits, light cleavage sites, as desired. In some embodiments, the surface of the TFM substrate includes two or more distinct adhesion protein domains. In such embodiments, the number may vary, ranging in some instances from 2 to 100,000, such as 5 to 50,000, including 10 to 10,000, e.g., 20 to 5,000, while in some instances the number of distinct adhesion protein domains ranges from 2 to 100, such as 2 to 50, e.g., 2 to 25. In some instances, the surface of the TFM substrate includes an array of adhesion protein domains.

Aspects of the invention further include a video imager configured to obtain video data from a hiPSC-CM present on the adhesion protein domain. By video imager it is meant a device or sensor, e.g., camera, capable of recording or obtaining video data. Any convenient video imager may be employed, where an example of a suitable video imager is provided in the Experimental section, below. In some instances the video data that is obtained by the video imager includes bright field data. Bright-field data is video data of the hiPSC-CM present on the adhesion protein domain when illuminated via bright-field microscopy, such as a white light. In some instances the video data that is obtained by the video imager includes fluorescence data. Fluorescence data is video data of fluorescent emissions (e.g., as specific wavelength) from the hiPSC-CM present on the adhesion protein domain when illuminated at an excitation wavelength, such as a light source of a wavelength absorbed by fluorophores in the TFM hydrogel, adhesion protein domain, or hiPSC-CM. In some instances, the video data that is obtained by the video imager includes both bright field and fluorescence data. As such, the video imager is configured to detect both bright field and fluorescent light emissions from the adhesion protein domain and any cell(s) present thereon. In some instances the video data includes three distinct types or channels of data, i.e., bright field data, a first set of fluorescence data (e.g., from fluorescent markers of the TFM substrate) and a second set of fluorescence data (e.g., from fluorescence markers on and/or inside of the cell). In such instances, the second set of fluorescence data differs from the first set in terms of detected wavelength, where the magnitude of detected wavelength difference between the two sets of data may vary, and in some instances ranges from 25 to 500 nm, such as 50 to 200 nm.

Systems of invention may further include one or more light (i.e., illumination) sources. Any convenient light source may be employed, where light sources of interest include lamps, lasers, LEDs, etc.

Systems of the invention further include a processing module configured to receive the video data and derive one or more parameters of an imaged hiPSC-CM therefrom. The nature of the parameter that is derived by the processing module may vary, where in some instances the processing module is configured to derive two or more distinct parameters, e.g., three, four, five or more parameters, as desired. In some embodiments, the processing module is configured to derive a contractile dynamic parameter. By contractile dynamic parameter is meant a parameter relating to the amount of stresses that each cell can generate during their contractile cycle, such as contractile force (ΣF). Contractile data that may be employed in determining a contractile parameter may vary, where such data may include synchronicity, movement velocity, time of contraction, electrical paceability, etc. In some embodiments, the processing module is configured to derive a kinetic dynamic parameter. By kinetic dynamic parameter is meant a parameter relating to the kinetic properties of the contractile cycle, such as beat rate, peak velocity of contraction (V_{C}), or peak velocity of relaxation (V_{R}). Mechanical data that may be employed in determining a mechanical parameter may vary, where such data may include force, work, power, etc. In some embodiments, the processing module is configured to derive a mechanical output parameter. By mechanical output parameter is meant a parameter that combines contractile and kinetic parameters, such as peak of contraction (P_{C}) or peak of relaxation (P_{R}). In some embodiments, the processing module is configured to derive a myofibril dynamic parameter. Myofibril dynamic data that may be employed in determining a myofibril dynamic parameter may vary, where such data may include sarcomere shortening, myofibril alignment, sarcomere registry, etc.

The processing module may be implemented using any convenient combination of hardware and/or software components. As would be recognized by one of skill in the art, many different hardware options and data structures can be employed to implement the processing module. Substantially any general-purpose computer can be configured to a functional arrangement for the methods and programs disclosed herein. The hardware architecture of such a computer is well known by a person skilled in the art, and can comprise hardware components including one or more processors (CPU), a random-access memory (RAM), a read-only memory (ROM), an internal or external data storage medium (e.g., hard disk drive). A computer system can also comprise one or more graphic boards for processing and outputting graphical information to display means. The above components can be suitably interconnected via a bus inside the computer. The computer can further comprise suitable interfaces for communicating with general-purpose external components such as a monitor, keyboard, mouse, network, etc. In some embodiments, the computer can be capable of parallel processing or can be part of a network configured for parallel or distributive computing to increase the processing power for the present methods and programs. In some embodiments, the program code read out from the storage medium can be written into a memory provided in an expanded board inserted in the computer, or an expanded unit connected to the computer, and a CPU or the like provided in the expanded board or expanded unit can actually perform a part or all of the operations according to the instructions of the program code, so as to accomplish the functions described below. In other embodiments, the method can be performed using a cloud computing system. In these embodiments, the data files and the programming can be exported to a cloud computer, which runs the program, and returns an output to the user.

The memory of a computer system can be any device that can store information for retrieval by a processor, and can include magnetic or optical devices, or solid state memory devices (such as volatile or non-volatile RAM). A memory or memory unit can have more than one physical memory device of the same or different types (for example, a memory can have multiple memory devices such as multiple drives, cards, or multiple solid state memory devices or some combination of the same). With respect to computer readable media, “permanent memory” refers to memory that is permanent. Permanent memory is not erased by termination of the electrical supply to a computer or processor. Computer hard-drive ROM (i.e., ROM not used as virtual memory), CD-ROM, floppy disk and DVD are all examples of permanent memory. Random Access Memory (RAM) is an example of non-permanent (i.e., volatile) memory. A file in permanent memory can be editable and re-writable.

In use, obtained data is input into and/or received by the processing module and the processing module outputs the one or more parameters that it is configured to determine, e.g., to a user.

In certain embodiments, instructions in accordance with the methods described herein can be coded onto a computer-readable medium in the form of “programming”, where the term “computer readable medium” as used herein refers to any storage or transmission medium (including non-transitory versions of same) that participates in providing instructions and/or data to a computer for execution and/or processing. Examples of storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer.

The computer-implemented method described herein can be executed using programming that can be written in one or more of any number of computer programming languages. Such languages include, for example, Java (Sun Microsystems, Inc., Santa Clara, Calif.), Visual Basic (Microsoft Corp., Redmond, Wash.), and C++ (AT&T Corp., Bedminster, N.J.), as well as any many others.

In some instances, the system further includes a positioner configured to place a hiPSC-CM on an adhesion protein domain of the TFM-hydrogel. Any convenient cellular positioning device may be employed, where positioning devices of interest include, but are not limited to: a micropipette, microfluidics channel, cell sorter and the like.

In some instances, e.g., where the systems are employed in active agent screening applications, the systems may include an introducer configured to contact an active agent (e.g., a drug, candidate drug, toxin, etc.) with a hiPSC-CM on an adhesion protein domain. Any convenient active agent introducing device may be employed, where active agent introducing devices of interest include, but are not limited to: a micropipette, microfluidics input channel, and the like. The active agent introduce may be one that selectively introduces an active agent to a specific cell on a specific adhesion protein domain, or one that contacts multiple cells on multiple protein adhesion domains at the same time.

In some instances, the systems may further be configured to remove a viable hiPSC-CM from an adhesion protein domain following video data acquisition, e.g., where a given protocol includes further analysis of the hiPSC-CM. In such embodiments, the system may include one or more components configured to release a hiPSC-CM from an adhesion protein domain, where such a component may include a mechanical separator, such as micropipette or a liquid flow modulator, a stimulus source, such as a source of a chemical or physical stimulus which releases cells from the adhesion protein domain, etc.

In some instances, the systems are configured as microfluidic systems. A “microfluidic device” system is a system that is configured to control and manipulate fluids geometrically constrained to a small scale (e.g., millimeter, sub-millimeter, etc.). Embodiments of the microfluidic devices may be made of any suitable material that is compatible with the assay conditions, samples, buffers, reagents, etc. used in the microfluidic device. In some cases, the microfluidic device is made of a material that is inert (e.g., does not degrade or react) with respect to the samples, buffers, reagents, etc. used in the subject microfluidic device and methods. For instance, the microfluidic device may be made of materials, such as, but not limited to, glass, quartz, polymers, elastomers, paper, combinations thereof, and the like.

In some instances, the microfluidic device includes one or more input ports. The input port may be configured to allow an assay constituent, e.g., a cell, a candidate active agent, etc., to be introduced into the microfluidic device. The input port may further include a structure configured to prevent fluid from exiting the sample input port. For example, the input port may include a cap, valve, seal, etc. that may be, for instance, punctured or opened to allow the introduction of a sample into the microfluidic device, and then re-sealed or closed to substantially prevent fluid, including the sample and/or buffer, from exiting the input port. In some instances, the microfluidic device includes one or more output ports. The output port may be configured to allow an assay constituent, e.g., a cell, a candidate active agent, etc., to be removed from the microfluidic device. The output port may further include a structure configured to prevent fluid from exiting the sample output port. For example, the output port may include a cap, valve, seal, etc. that may be, for instance, punctured or opened to allow the removal of a cell the microfluidic device, and then re-sealed or closed to substantially prevent fluid, including the sample and/or buffer, from exiting the output port.

Positioned between, and fluidically coupled to, the input and output ports may be one or more chambers that include a TFM substrate, e.g., as described above, where the TFM substrate is operatively coupled to the light source and video imager, e.g., as described above. In some instances, the device may include a single chamber with a TFM substrate, which substrate may include an array of adhesion protein domains, e.g., for binding to an array of cells. In yet other embodiments, the device may include multiple chambers, where each of multiple chambers may include one or more adhesion protein domains for binding to cells.

In certain embodiments, the microfluidic device is substantially transparent. By “transparent” is meant that a substance allows visible light to pass through the substance. In some embodiments, a transparent microfluidic device facilitates analysis of cell(s) in the device. In some cases, the microfluidic device is substantially opaque. By “opaque” is meant that a substance does not allow visible light to pass through the substance.

In certain embodiments, the microfluidic device has a width ranging from 10 cm to 1 mm, such as from 5 cm to 5 mm, including from 1 cm to 5 mm. In some instances, the microfluidic device has a length ranging from 100 cm to 1 mm, such as from 50 cm to 1 mm, including from 10 cm to 5 mm, or from 1 cm to 5 mm. In certain aspects, the microfluidic device has an area of 1000 cm² or less, such as 100 cm² or less, including 50 cm² or less, for example, 10 cm² or less, or 5 cm² or less, or 3 cm² or less, or 1 cm² or less, or 0.5 cm² or less, or 0.25 cm² or less, or 0.1 cm² or less.

Any convenient microfluidic device architecture may be employed. Representative architectures that may be modified to be employed in systems of the invention include, but are not limited to, those described in: U.S. Pat. Nos. 9,738,887; 9,657,341; 9,322,054; 9,205,396; 9,156,037; and 8,911,989; as well as U.S. Pat. Nos. 9,498,776; 9,103,825; and 9,039,997; United States Published Patent Application Nos.: 20140342445; 20130230881 and 20110129850; as well as Published PCT Application Publication No. WO/2015/013210, the disclosures of which are herein incorporated by reference. Microfluidic devices and systems that may be adapted for the present invention further include those described in: Fang et al., Anal. Chim Acta (2016) 903:36-50; Ahn et al., Methods. Mol. Biol. (2014) 1185:223-33; Ertl et al., Trends Biotechnol. (2014) 32: 245-53; Cosson et al., Sci. Rep. (2014) 25: 4:4462; Titmarsh et al., Stem Cells Transl. Med. (2014) 3: 81-90; Mahadik et al., Adv. Healthc. Mater. (2014) 3: 449-458; Zhang et al., Bionanoscience (2012) 1:277-286; Kim et al., Lab Chip (2011) 7:104-14; and Mathur et al., Scientific Reports (2015) 5: 8883.

In some embodiments, the output of the microfluidic device is operably coupled to a cell analyzer, such that the output of the microfluidic device delivers a retrieved hiPSC-CM to a cell analyzer device. Cell analyzer devices that may be operably coupled to the retriever may vary, as desired, where examples of such devices include, but are not limited to: flow cytometers, nucleic acid analysis (e.g., qPCR) platforms, protein analysis platforms, mass cytometers, and the like.

In some instances, the cell analyzer is a flow cytometer. Flow cytometry is a methodology using multi-parameter data for identifying and distinguishing between different particle (e.g., cell) types i.e., particles that vary from one another in terms of label (wavelength, intensity), size, etc., in a fluid medium. In flow cytometrically analyzing a sample, an aliquot of the sample is first introduced into the flow path of the flow cytometer. When in the flow path, the cells in the sample are passed substantially one at a time through one or more sensing regions, where each of the cells is exposed separately and individually to a source of light at a single wavelength (or in some instances two or more distinct sources of light) and measurements of cellular parameters, e.g., light scatter parameters, and/or marker parameters, e.g., fluorescent emissions, as desired, are separately recorded for each cell. The data recorded for each cell is analyzed in real time or stored in a data storage and analysis means, such as a computer, for later analysis, as desired.

In flow cytometry-based methods, the cells are passed, in suspension, substantially one at a time in a flow path through one or more sensing regions where in each region each cell is illuminated by an energy source. The energy source may include an illuminator that emits light of a single wavelength, such as that provided by a laser (e.g., He/Ne or argon) or a mercury arc lamp or an LED with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 405 nm, 535 nm, 561 nm, 635 nm, 642 nm, and the like. Following excitation of a labeled specific binding member bound to a polypeptide by an energy source, the excited label emits fluorescence and the quantitative level of the polypeptide on each cell may be detected, by one or more fluorescence detectors, as it passes through the one or more sensing regions.

In flow cytometry, in addition to detecting fluorescent light emitted from cells labeled with fluorescent markers, detectors, e.g., light collectors, such as photomultiplier tubes (or “PMT”), an avalanche photodiode (APD), etc., are also used to record light that passes through each cell (generally referred to as forward light scatter), light that is reflected orthogonal to the direction of the flow of the cells through the sensing region (generally referred to as orthogonal or side light scatter) as the cells pass through the sensing region and is illuminated by the energy source. Each type of data that is obtained, e.g., forward light scatter (or FSC), orthogonal light scatter (SSC), and fluorescence emissions (FL1, FL2, etc.), comprise a separate parameter for each cell (or each “event”).

Flow cytometers may further include one or more electrical detectors. In certain embodiments, an electrical detector may be employed for detecting a disturbance caused by a particle or cell passing through an electrical field propagated across an aperture in the path of the particles/cells. Such flow cytometers having electrical detectors will contain a corresponding electrical energy emitting source that propagates an electrical field across the flow path or an aperture through which cells are directed. Any convenient electrical field and/or combination of fields with appropriate detector(s) may be used for the detection and/or measurement of particles (or cells) passing through the field including but not limited to, e.g., a direct current electrical field, alternating current electrical field, a radio-frequency field, and the like.

Flow cytometers further include data acquisition, analysis and recording means, such as a computer, wherein multiple data channels record data from each detector for each cell as it passes through the sensing region. The purpose of the analysis system is to classify and count cells wherein each cell presents itself as a set of digitized parameter values and to accumulate data for the sample as a whole.

A particular cell subpopulation of interest may be analyzed by “gating” based on the data collected for the entire population. To select an appropriate gate, the data is plotted so as to obtain appropriate separation of subpopulations, e.g., by adjusting the configuration of the instrument, including e.g., excitation parameters, collection parameters, compensation parameters, etc. In some instances, this procedure is done by plotting forward light scatter (FSC) vs. side (i.e., orthogonal) light scatter (SSC) on a two dimensional dot plot. The flow cytometer operator then selects the desired subpopulation of cells (i.e., those cells within the gate) and excludes cells which are not within the gate. Where desired, the operator may select the gate by drawing a line around the desired subpopulation using a cursor on a computer screen. Only those cells within the gate are then further analyzed by plotting the other parameters for these cells, such as fluorescence.

Any flow cytometer that is capable of obtaining fluorescence data, e.g., as described above, may be employed. Useful flow cytometers include those utilizing various different means of flowing a cell through the sensing region substantially one at a time including, e.g., a flow cell, a microfluidics chip, etc. Non-limiting examples of flow cytometer systems of interest are those available from commercial suppliers including but not limited to, e.g., Becton-Dickenson (Franklin Lakes, N.J.), Life Technologies (Grand Island, N.Y.), Acea Biosciences (San Diego, Calif.), Beckman-Coulter, Inc. (Indianapolis, Ind.), Bio-Rad Laboratories, Inc. (Hercules, Calif.), Cytonome, Inc. (Boston, Mass.), Amnis Corporation (Seattle, Wash.), EMD Millipore (Billerica, Mass.), Sony Biotechnology, Inc. (San Jose, Calif.), Stratedigm Corporation (San Jose, Calif.), Union Biometrica, Inc. (Holliston, Mass.), Cytek Development (Fremont, Calif.), Propel Labs, Inc. (Fort Collins, Colo.), Orflow Technologies (Ketchum, Id.), handyem inc. (Québec, Canada), Sysmex Corporation (Kobe, Japan), Partec Japan, Inc. (Tsuchiura, Japan), Bay bioscience (Kobe, Japan), Furukawa Electric Co. Ltd. (Tokyo, Japan), On-chip Biotechnologies Co., Ltd (Tokyo, Japan), Apogee Flow Systems Ltd. (Hertfordshire, United Kingdom), and the like.

### Methods

Also provided are methods for assaying cardiomyocytes, such as human induced pluripotent stem cell-derived cardiomyocytes (hiPSC-CMs), e.g., using a system such as described above. Aspects of the methods include positioning a hiPSC-CM on an adhesion protein domain present on a surface of a traction force microscopy hydrogel (TFM-hydrogel); obtaining video data from the hiPSC-CM present on the adhesion protein domain; and deriving a parameter of the hiPSC-CM from the obtained video data.

The hiPSC-CM may be positioned on the adhesion protein domain using any convenient protocol. In some instances, the hiPSC-CM is positioned on the adhesion protein domain by a micropipette, microfluidics input channel, cell sorter, and the like. For example, an initial liquid sample of hiPSC-CMs may be flowed over a TFM substrate of the invention such that cells adhere to adhesion domains of the TFM substrate. Following position of cells on the TFM substrate, the cells may be allowed to grow to obtain desired properties, e.g., phenotypes that resemble mature adult cardiomyocytes, such as myofibril alignment and sarcomere registry as seen in adult CMs, beating properties, etc. While this culture stage may vary in length, in some instances the cells are cultures for a period of 1 to 40 days, such as 5 to 30 days, e.g., 10 to 20 days.

When the cells on the TFM substrate have achieved desired phenotypes, e.g., phenotypes resembling adult CMs, the cells may be contacted with one or more labeling reagents, as desired. For example, the cells may be contacted with an actin dynamic visualization reagent, e.g., for obtaining myofibril dynamic data during. Examples of actin dynamic visualization reagents that may be employed include, but are not limited to: fluorescently labeled actin, actin-GFP nucleic acid encoding reagents, fluorescent-protein/actin binding domain fusion proteins and nucleic acids encoding the same; and Lifeact (Riedl et al., Lifeact: a versatile marker to visualize F-actin,” Nat. Methods (2008) 5:605.).

Following placement of the hiPSC-CM on the adhesion protein domain and any desired labeling thereof, e.g., as described above, video image data of the hiPSC-CM is obtained. The video image data that is obtained may vary, and may include bright field and/or fluorescence video data. In some instances, the video image data includes bright field data and fluorescence data at two or more wavelengths or channels (e.g., one of the TFM substrate fluorescent marker and one for a fluorescent cellular label, e.g., Lifeact). The data is obtained for a duration of time, where the duration of time may also vary, ranging in some instances from 1 second to 60 seconds, such as 4 seconds to 10 seconds.

Following obtainment of the video image data, one or more parameters of the hiPSC-CM is derived from the obtained video image data, e.g., by using a processing module as described above. The one or more parameters that are derived may vary, where examples of parameters that may be derived include, but are not limited to: peak displacement (dₘₐₓ), peak force (ΣFₘₐₓ), peak velocity of contraction (V_{C}), peak velocity of relaxation (V_{R}), peak power of contraction (P_{C}) and peak power of relaxation (P_{R}). Any convenient algorithm may be employed to obtain the one or more parameters, where examples of algorithms that may be employed are described in the Experimental section, below.

In some embodiments, the methods include assessing the impact of an active agent on the hiPSC-CM, e.g., where a candidate agent is screened for therapeutic activity. Screening assays of interest include methods of assessing whether a test compound modulates one or more hiPSC-CM parameters in some way. By “assessing” is meant at least predicting that a given test compound will have a given (e.g., desirable) activity, such that further testing of the compound in additional assays, such as animal model and/or clinical assays, is desired. Drug screening may be performed by contacting a hiPSC-CM in a system of the invention and then assessing the activity of the agent based on its modulation of the cell. As such, methods of invention may include contacting a cell on an adhesion protein domain of a TFM substrate of a system with an active agent whose activity is to be screened. Contact may be achieved using any convenient protocol, such as contacting an entire TFM substrate surface with a solution of the active agent, selectively contacting a protein adhesions domain with a solution of the active agent, etc.

The term “agent” as used herein describes any molecule, e.g., protein or pharmaceutical. In some embodiments, a plurality of assay mixtures are run in parallel with different agent concentrations to obtain a differential response to the various concentrations. In such instances, one of these concentrations serves as a negative control, i.e., at zero concentration or below the level of detection. Candidate agents encompass numerous chemical classes, such as organic molecules, e.g., small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Of interest in certain embodiments are compounds that pass the blood-brain barrier. Where the screening assay is a binding assay, one or more of the molecules may be joined to a member of a signal producing system, e.g., a label, where the label can directly or indirectly provide a detectable signal. Various labels include, but are not limited to: radioisotopes, fluorescers, chemiluminescers, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used.

The compounds having the desired pharmacological activity may be administered in a physiologically acceptable carrier to a host for treatment or prevention of a disease. The agents may be administered in a variety of ways, orally, topically, parenterally e.g., subcutaneously, intraperitoneally, by viral infection, intravascularly, etc. Depending upon the manner of introduction, the compounds may be formulated in a variety of ways. The concentration of therapeutically active compound in the formulation may vary from about 0.1-10 wt %.

In some instances, the methods include retrieving the hiPSC-CM from the adhesion protein domain. A hiPSC-CM may be retrieved from the adhesion protein domain using any convenient protocol. In some instances, the hiPSC-CM is retrieved from the adhesion protein domain via a mechanical protocol, e.g., by mechanically removing the cell with a device, such as a micropipette, by mechanically removing the cell using flowing liquid, etc. In some instances, a stimulus may be employed to separate a cell from a domain, such as a chemical stimulus, enzymatic stimulus, light stimulus (e.g., where the cell is adhered to the TFM substrate via a light cleavable adhesion molecule), etc.

In some embodiments, the methods further include analyzing the retrieved hiPSC-CM. Retrieved hiPSC-CMs may be further analyzed using any convenient protocol. Protocols of interest to which retrieved hiPSC-CMs may be subjected include, but are not limited to: calcium ion signaling assays, nucleic acid analysis (e.g., PCR, qRT-PCR, etc.), protein analysis (e.g., immunocytochemistry), flow cytometry, mass cytometry, and the like. The obtained data from downstream analysis of retrieved hiPSC-CMs may be matched with the video data derived parameter(s) obtained for the hiPSC-CMs, as desired.

### Kits

Also provided are kits that at least include the subject systems and which may be used according to the subject methods, e.g., as described above. The kits may further include one or more components to be employed in a given protocol, e.g., tools, reagents for harvesting and/or preparing hiPSC-CMs, etc. The components of the kits may be present in sterile packaging, as desired.

In certain embodiments, the kits which are disclosed herein include instructions, such as instructions for using devices. The instructions for using devices are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging etc.). In other embodiments, the instructions are present as an electronic storage data file present on a suitable computer readable storage medium, e.g., Portable Flash drive, CD-ROM, diskette, etc. The instructions may take any form, including complete instructions for how to use the device or as a website address with which instructions posted on the world wide web may be accessed.

The following examples are provided by way of illustration and not by way of limitation.

### Experimental

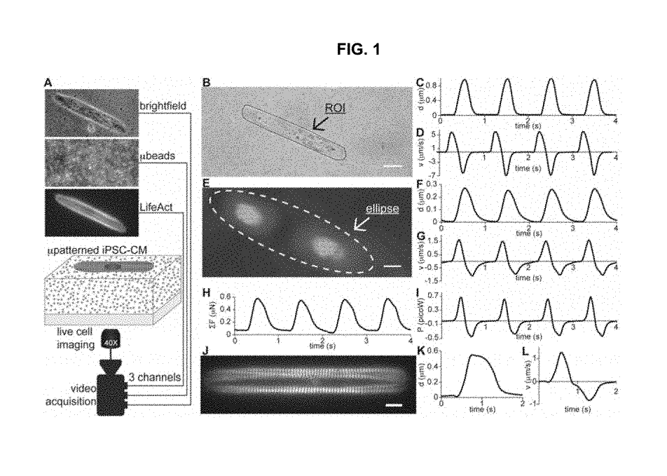

Here we present a computational platform that integrates different methods to analyze the mechanical output of μpatterned hiPSC-CMs (FIG. 1). Our platform analyzes bright field videos of single beating cells, videos of the substrate moving due to cell-generated tractions and videos of labeled myofibrils. The output is a set of contractile and kinetic parameters that characterize the mechanical output of μpatterned hiPSC-CMs. We also present novel approaches to measure sarcomere length from videos of moving myofibrils and to quantify the synchronicity of contractile movement within a single cell. This analytical platform detected drug-induced effects on the mechanical output of μpatterned hiPSC-CMs, as well as contractile defects due to decreased expression of the myosin binding protein C gene (MYBPC3), thus validating its ability to assay for cardiac contractile function.

### I. METHODS

Fabrication of Matrigel Micropatterns on Polyacrylamide Substrates

We cultured single hiPSC-CMs on Matrigel micropatterns, which were transferred from printed glass coverslips onto the surface of polyacrylamide hydrogels with a stiffness of 10 kPa as previously described. (Ribeiro 2015a) In summary, Matrigel was diluted 1:10 in L15 medium (Thermo Fisher) and added to the top of elastomeric microstamps composed of polydimethylsiloxane 182 (Dow Corning) to be incubated at 3-4° C. overnight. Microstamps consisted of 2000 μm² rectangular features with an aspect ratio of 7:1 (length:width). We gently aspirated Matrigel after the overnight incubation, washed the stamps twice in L15 medium, aspirated L15 medium from the surface and dried it with a low stream of N₂ gas. We then used microstamps to micropattern the Matrigel rectangular features on clean glass coverslips by microcontact printing. Matrigel on micropatterns was transferred to the surface of the polyacrylamide substrates during the gelation process by placing the patterned coverslip in contact with the top of the acrylamide prepolymer solution right before gelation. The aqueous prepolymer solution to be gelled was composed of acrylamide (Sigma-Aldrich)(10% w/v), bisacrylamide (Sigma-Aldrich) (0.1% w/v), ammonium persulfate (Sigma-Aldrich) (0.01% w/v) and N,N,N′,N′-tetramethylethylenediamine (Sigma-Aldrich) (0.1% v/v), HEPES (Thermo Fisher) (35 mM) and Milli-Q water. To calculate the forces generated by cells attached to polyacrylamide surfaces with traction force microscopy, green fluorescent microbeads with a diameter of 0.2 μm (Thermo Fisher) were also dispersed in the gel solution to yield a final concentration of 6.25×10⁹ microbeads/mL. We gelled acrylamide on top of another coverslip functionalized with 3-(trimethoxysilyl)propyl methacrylate (Sigma-Aldrich), which binds polyacrylamide. In the end of this process, the polyacrylamide substrates remained attached to the bottom glass coverslip and did not freely float or swell after gelation, which was key for maintaining cells in culture and imaging them. Once polymerized, polyacrylamide substrates were incubated in PBS for at least 2 hours and the top coverslips were carefully removed with a razor blade. We seeded hiPSC-CMs on these polyacrylamide hydrogel substrates after washing them 3 times with PBS.

Differentiation, Culture and Seeding of hiPSC-CMs

We differentiated human induced pluripotent stem cells (hiPSCs) into monolayers of spontaneously beating cardiomyocytes (hiPSC-CMs) with a small-molecule-mediated, Wnt-modulating protocol. (Lian 2013) We increased the percentage of differentiated hiPSC-CMs in culture by using lactate instead of glucose as the carbon source, (Tohyama 2013). Once differentiated around day 20-25, we froze cells for later use with a freezing medium composed of fetal bovine serum (Thermo Fisher) with 10 μM Y27623 ROCK inhibitor (Stemcell Technologies) and 10% dimethyl sulfoxide (Sigma-Aldrich).

Before transferring cells onto micropatterns on polyacrylamide gel devices, we thawed cells into wells of six-well culture plates coated with fibronectin from bovine serum (Sigma) and cultured cells in RPMI-1640 medium containing B27 supplement (50×), penicillin (25 μg/mL) and streptomycin (50 μg/mL) (all from Thermo Fisher) with 5 μM Y27623 ROCK inhibitor. 2 days after thawing cells, we added fresh culture medium without ROCK inhibitor and allowed cells to recover from thawing for 2 more days before passaging them to polyacrylamide devices, when hiPSC-CMs should be spontaneously beating within a semi-confluent monolayer.

We passaged hiPSC-CMs onto micropatterned polyacrylamide devices at a density of 1000 cells/cm². For this purpose, cultures of thawed cells were washed twice with PBS and incubated in 1 mL of Accutase for 8-10 min. After observing cell detachment from the bottom of the wells, we quenched Accutase with Dulbecco's modified Eagle's medium (Thermo Fisher) containing 12% fetal bovine serum and counted the concentration of cells in medium with a hemocytometer. Then, we centrifuged cells at 83 rcf for 3 min at room temperature and aspirated the supernatant to resuspend the pelleted cells in the required volume of medium (RPMI-1640 cell-culture medium with 5 μM ROCK inhibitor) for adding 150 μL of cell solution to the surface of hydrogel devices at a concentration of 1000 cells/cm². After 1.5 h of incubation, we added 2.5 mL of medium to the well containing hydrogel devices and added fresh medium without ROCK inhibitor after 2 days. Single beating cells on Matrigel patterns were analyzed between 5-10 days after seeding.

Imaging, Labeling and Pharmacological Stimulation of Live hiPSC-CMs

We imaged the movement of beating hiPSC-CMs and the displacement of fluorescent microbeads embedded in polyacrylamide substrates with a Zeiss Axiovert 200M inverted microscope equipped with a Zeiss Axiocam MRm CCD camera. This microscope also contained an environmental chamber (PeCon) to set temperature at 37° C. Unless otherwise noted, we acquired microscopy videos while electrically pacing hiPSC-CMs with 10 ms-wide bipolar pulses of electric-field stimulation at 10-15 V with a frequency of 1 Hz (Myopacer, lonOptix).

We fluorescently labeled actin with LifeAct in live hiPSC-CMs to image myofibrils and sarcomeres as described before. (Ribeiro 2015a) For this effect, we incubated hiPSC-CMs with the adenovirus rAV CAG-LifeAct-TagRFP (Ibidi; 1×10⁵ IU/mL in cell-culture medium) overnight at 37° C. in a humid 5% CO2 atmosphere. We then washed cells once with PBS at 37° C. and added new medium. We observed actin labeled in sarcomeres after 2 days of adding the adenovirus to the culture medium.

To induce contractile changes in hiPSC-CMs, we added drugs known to affect CM contractile machinery to the culture medium. We analyzed the mechanical output of cells after adding drugs. Caffeine (Sigma-Aldrich, Saint Louis, Mo.) was added to achieve a final concentration of 10 mM. We exposed single cells to concentrations of 0.1 μM and then 1 μM of isoproterenol (Sigma, Saint Louis, Mo.) to test how the contractile activity of each cell varied after adding different amounts of isoproterenol to the medium. Omecamtiv mecarbil (Adooq Bioscience, Irvine, Calif.) was also added to the cell culture medium at 10 nM or 0.1 μM to detect different variations in the contractile response of hiPSC-CMs after adding one of these concentrations.

Characterization of Cell Movement as a Phenotype of Mechanical Output

We acquired videos of single beating hiPSC-CMs imaged with differential interference contrast microscopy at frame rates higher than 30 fps for a time between 4 and 10 s. We used MATLAB (R2014b version, Mathworks) to convert pixels of each frame to microns. Zeiss files (.czi) contained information on the pixel dimensions and were imported into MATLAB using the Bioformats package. (Linkert 2010) We also confirmed the software calibration with calibration slides (Electron Microscopy Sciences). For each video of a beating single cell, we selected a region of interest (ROI) around the borders of the cell and analyzed average displacements within this ROI (FIG. 1B). For this purpose, we selected a baseline frame, within all the frames of the video, which represented the relaxed state of the cell and calculated average displacement within the ROI for the remaining frames relative to the baseline frame. As noted bellow, this procedure was automated. We then calculated displacements from microscopy videos with cross-correlation approaches: digital image correlation (DIC) and particle image velocimetry (PIV) approaches. (McCormick 2010, Adrian 2005) The outputs of these analyses were plots that showed how the average displacement within the ROI varied in time during the different contractile cycles of a beating hiPSC-CM (FIG. 1). Cross-correlation is done between sub-blocks of the image and the highest correlation indicates the maximum likelihood that blocks match. We tested the following cross-correlation algorithms: PIVIab, ImageJ PIV (Tseng 2012) and Ncorr. PIVIab and Ncorr were written in MATLAB, while ImageJ PIV is a plugin for ImageJ (NIH). Ncorr generally performs better, but uses more computational time. (Blaber 2015)

The average displacement in the ROI was defined as

$$d ( {t}_{k} ) = \frac{1}{N} {\sum }_{k = 1}^{N} \sqrt{{u}_{k , x}^{2} + {u}_{k , y}^{2}} , \qquad ( 1 )$$

where k=1, . . . , N corresponds to the frame number of the video and (uₓ,u_{y})^{T} to the displacement vector at each discrete pixel point inside of the ROI. The maximal contraction displacement was then defined as the total distance between the fully relaxed and fully contracted states of the cell's contractile cycle. We calculated such distance by subtracting the minima by the maxima of d(t). Minima of d(t) represent detected noise, which was approximately constant. Maximal contraction displacement was then defined as

$${d}_{c} = \frac{1}{m} {\sum }_{i = 1}^{m} {max}_{i} ( d ( {t}_{k} ) ) - \frac{1}{n} {\sum }_{j = 1}^{n} {min}_{j} ( d ( {t}_{k} ) ) . \qquad ( 2 )$$

The different detected maxima were defined by m and the different minima were defined by n. Maxᵢ and minⱼ are the local maximum and local minimum values for each contraction cycle in the d-curve. Once average displacement within the ROI is plotted as a function of time, we also plotted average velocity of movement within the ROI (FIG. 1) by calculating the first derivative of the mean movement as

$$V ( {t}_{k} ) \approx \frac{Δ d}{Δ t} = \frac{{d}_{k + 1} - {d}_{k - 1}}{{t}_{k + 1} - {t}_{k - 1}} , \qquad ( 3 )$$

where dₖ is an abbreviation for d(tₖ). We then calculated for each contractile cycle the maximal velocity of contraction (V_{C}) and the maximal velocity of relaxation (V_{R}) from the velocity plot (FIG. 1) (Ribeiro 2014) as follows:

$${V}_{C} = \frac{1}{m} {\sum }_{i = 1}^{m} {max}_{i} ( V ( {t}_{k} ) ) ; {V}_{R} = \frac{1}{n} {\sum }_{j = 1}^{n} {min}_{j} ( V ( {t}_{k} ) ) . \qquad ( 4 )$$

Also here, m corresponds to the total maxima, n corresponds to the total minima and maxᵢ and minᵢ respectively represent local maximum and local minimum values for each contraction cycle in the V-curve. Beat rate (br) is defined as the number of times a cell undertakes contractile cycles per unit of time. We used two different approaches to determine f from the curve d(tₖ). In the first one, Fourier transformation of d(tₖ) exhibited dominant peaks, which corresponded to main frequencies. In the second approach, periods were selected on the d(tₖ) curve in the time domain and then f was calculated as the inverse of period (T). T was defined as the time between adjacent peaks of d(tₖ). After determining different values of T for each adjacent peak to d(tₖ), f was calculated as:

$$br = {( \frac{1}{m} {\sum }_{i = 1}^{m} {T}_{i} )}^{- 1} . \qquad ( 5 )$$

For periodic functions, such as d(tₖ), both approaches deliver the same result.

The second approach offers higher flexibility when d(tₖ) has high levels of noise, which may affect the calculation off through Fourier transformation. In addition, if cell beating does not occur periodically there can be multiple dominant frequencies (i.e. peaks in Fourier space). Picking periods allows the selection of individual and clearly discernible periodic motion.

To determine the time of duration of each contraction, we calculated the time at which velocity is highest in contraction and the time at which velocity is highest in relaxation. (Ribiero 2014) We then calculated the temporal distance between each adjacent maximum and minimum on the plot of v(tₖ) and averaged for all contraction cycles

$$\hat{t} = \frac{1}{m} {\sum }_{i = 1}^{m} ‖ {t}_{k} \vert {max}_{i} V ( {t}_{k} ) - {t}_{k} \vert {min}_{i} V ( {t}_{k} ) ‖ . \qquad ( 6 )$$

The value of m represents the number of contraction cycles. Ideally, the time of each contractile cycle could be calculated from d(tₖ). However, this approach is difficult because the exact beginning and end of each contraction curve are often hard to select with exactitude. Determining \hat{t} has the advantage of being less biased than determining the total time of contraction because its calculation is clearly defined and always applied in the same way.

We measured two parameters of asynchronicity of contractile movement within the ROI: spatial asynchronicity (a_{θ}) of contractile movement and temporal asynchronicity (a_{δ}) of contractile movement. Spatial synchronicity occurs when the different regions of the cell move along the same directions and temporal synchronicity occurs when all the regions of the cell move at the same time. To calculate a_{θ}, we first computed the direction of movement (θ) for every movement vector of different regions i inside the ROI (FIG. 1B) at each frame as defined by

$${θ}_{i} = atan \frac{{v}_{i}}{{u}_{i}} , \qquad ( 7 )$$

where uᵢ and vᵢ are respectively the horizontal and vertical components of the displacement vectors. We expected a normal distribution of the different values of 8.

If all regions within the ROI move along the same direction during contractility, values of θ will not vary among the different regions i and the standard deviation of different θᵢ values will be low. However, asynchronous movement between different ROI regions i will originate high standard deviations of θᵢ. Therefore, to quantify a_{θ}, we calculated the standard deviation of θ as a measure of how asynchronous is displacement within the ROI:

a_{θ}=mean([std(θ)](t)). (8)

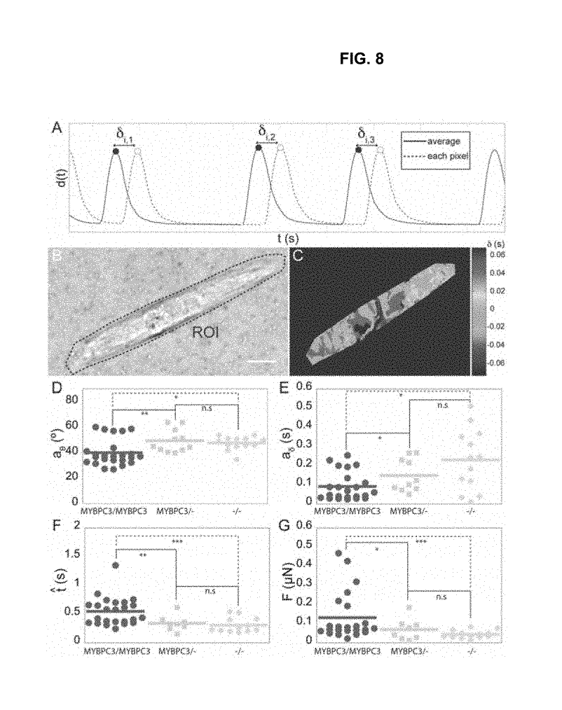

For the calculation of a_{δ}, we first determined the mean offset time b between the peaks of the displacement curves of different regions i (d_{current}) in the ROI and the peaks of the mean displacement (dₘₑₐₙ) that occurs within the same ROI by mean, cross-correlation between the two curves, then refined this offset result using main peaks only (FIG. 8A):

$$\begin{matrix}{δ}_{i} = \frac{1}{{N}_{peaks}} {\sum }_{j = 1}^{{N}_{peaks}} {δ}_{i , j} , & ( 9 ) \\ {δ}_{i , j} = {t}_{max} ( {d}_{current} ) - {t}_{max} ( {d}_{mean} ) . & ( 10 )\end{matrix}$$

When the timing of contractile movement of one region is synchronous with the timing of contractile movement of a reference region, values of δ are very small. However, δ will increase if the contractile movement is asynchronous between two zones in the ROI (FIG. 8C). Therefore, a_{δ} was obtained from the standard deviation of time offsets δ between different regions that compose the ROI:

a_{δ}=mean([std(δ)](t)). (11)

If cell beating is spatially and temporally synchronous within the selected ROI, a_{θ} and a_{δ} will have lower values than if the beating is less synchronous. Displacement fields for each individual frame were calculated with respect to a reference frame. The reference frame was not necessarily the first frame of the video and needs to be selected, but instead a frame that showed the cell in its most relaxed state. We did not control the contractile state of the cell to match the beginning of video acquisition to a relaxed state of a beating cell and therefore one did not know a priori the phase of the contractile cycle at which the cell was in the first frame of each video. The first frame of the video could represent a cell that is fully contracted, fully relaxed or in between those states. We selected a reference frame at which the cell was in a relaxed state. The choice of the reference frame was critical for the shape of d-curves and V-curves, as noted in FIG. 1. We automated the selection of the reference frame at which the cell is fully relaxed. In our automated algorithm for selecting the reference frame (frame_{ref}), the first frame of the video was initially selected as a first possible reference frame and compared with other frames to select the best one that can satisfy the following criteria:

$$\begin{matrix}maximize [ m ( {frame}_{ref} ) = Δ d \approx {{d}_{i} ( {t}_{{frame}_{ref}} )}_{max} - {{d}_{k} ( {t}_{{frame}_{ref}} )}_{min} ] , & ( 12 ) \\ minimize [ n ( {frame}_{ref} ) = {\sum }_{i = 1}^{N} ( {d}_{i} ( {t}_{{frame}_{ref}} ) ) ] . & ( 13 )\end{matrix}$$

To select the frame_{ref} the first criterion assured a maximal difference between the maximum and minimum points of the displacement curve. However, the solution for this criterion could be the desired frame_{ref} where the cell is in its most relaxed state, or a frame_{ref} where the cell is in its most contracted state. Reference frames between both states were excluded according to the first criterion. The second criterion selected a frame_{ref} that minimizes the area under the displacement curve, which satisfied the conditions for the d-curves presented in FIG. 1. These two criteria were enough to automatically smart-guess the frame_{ref} as a frame where the cell is in its most relaxed state. In addition, all smart-guess of the frame_{ref} was submitted to user-review of the resultant contraction d-curve. To robustly smart-guess the frame_{ref}, videos had to be acquired at a speed (frames per second) that could capture the cell at different stages of the contractile cycle. Smart-guessing the frame_{ref} also required an image resolution (μm/pixel) that allowed tracking movement within the ROI and low image noise. Once the frame_{ref} was selected, all displacements in regions within the ROI were calculated relative to frame_{ref}.

Traction Force Microscopy and Phenotypes of Mechanical Output

We estimated forces generate by iPSC-CMs with a traction force microscopy algorithm. Traction force microscopy estimates forces generated by adherent cells on deformable substrates. (Munevar 2001, Ribiero 2015b) This approach involves two distinct steps: i) measure the deformation of the substrate induced by cell-generated tractions and ii) derive forces from substrate deformations while considering the material mechanical properties of the substrate: Young's modulus (E) and Poisson's ratio (v).

As already noted in the fabrication section above, (Ribeiro 2015a) we dispersed fluorescent microbeads in the core of polyacrylamide hydrogels and quantified their displacement during contractions of hiPSC-CMs to track cell-induced deformations on polyacrylamide substrates. For this purpose, while cells were beating, we acquired videos of moving microbeads at frame rates above 25 fps and submitted these videos to cross-correlation particle tracking tools detailed in the beginning of the previous section. We also tracked displacement of microbeads as defined in the previous section to determine displacement curves d(t), the maximal velocity of contraction (V_{C}), the maximal velocity of relaxation (V_{R}), the beat rate (br) and the time between each adjacent maximum and minimum on the velocity plot (\hat{t}) (FIG. 1).

After quantifying cell-induced displacements on the surface of hydrogels, we estimated the traction stresses σ associated to each displacement vector of the surface and calculated force f for each stress vector. We then calculated the absolute value (F) of f for each pixel, which has a positive value independently of its orientation or coordinates. We summed the different values of F (ΣF) to calculate the total amount of force that each cell can generate on its extracellular environment during each contractile cycle. (Ribeiro 2015a)

Ahead we detail how Σ F is calculated from displacement fields of microbeads. As we plotted calculated values of ΣF as a function of time, we also plotted contractile power (P), which was calculated by multiplying ΣF by the velocity of movement of microbeads at each time point represented by the different video frames. From the curve P(t), we calculated the maximal power of contraction (P_{C}) and the maximal power of relaxation (P_{R}) (FIG. 1).

After determining displacements of moving microbeads from acquired videos, we estimated ΣF from these cell-induced displacements with traction force microscopy. (Dembo 1996) The continuum mechanics equations for linear elastic materials are described through force equilibrium conditions,

σ_{ji,j}+fᵢ=0, (14)

the material constitutive relations,

$${σ}_{ij} = \frac{E}{1 + υ} [ {ɛ}_{ij} + \frac{υ}{1 + 2 υ} {ɛ}_{kk} {δ}_{ij} ] , \qquad ( 15 )$$

and kinematic equations,

εᵢⱼ=½(u_{i,j}+u_{j,i}), (16)

where σ is the stress tensor, f is a force of external origin, E is the linear strain tensor and u is the displacement field. E is the Young's modulus of the polyacrylamide substrate and v is its Poisson's ratio. E and v are constants that depend on the properties of the deformable material. For polyacrylamide substrates, E is tunable in the kPa range (Wen 2014) and v is around 0.45 for thin polyacrylamide sheets for cell culture. (Kandow 2007) Equations 14 to 16 correspond to 15 equations with 15 unknowns that are expressed in a condensed form using the Einstein summation convention and the Kronecker delta δᵢⱼ (δᵢⱼ=0 if i≠j, δᵢⱼ=1 if i=j). One should note that the Kronecker delta δᵢⱼ is not related to the variable defined in equations 9 and 10, but uses the same notation.

These equations are valid while assuming that strains are small and linear and that the polyacrylamide substrate has homogeneous properties and behaves as an elastic solid. (Schwarz 2015) These assumptions satisfy the need for geometric linearity of strain and material linearity of the substrate.

By combining the governing equations, the balance of internal forces described in equation 14 can be written as a partial differential equation for the displacement vector field:

$$\frac{E}{2 ( 1 + v )} [ ( {u}_{j , ij} + {u}_{i , jj} ) + \frac{2 v}{1 - 2 v} {u}_{k , ij} {δ}_{ji} ] + {f}_{i} = 0. \qquad ( 17 )$$

Following the same procedure as Dembo and colleagues (Dembo 1996) and Landau and colleagues, (Landau 1986) while analyzing displacements of microbeads, we considered that cell-generated deformations on a polyacrylamide surface occurred in a semi-infinite elastic medium with a planar traction distribution on its surface. Specifically for a semi-infinite elastic medium bounded by a planar surface at z=0, we used a derivation of the Boussinesq solution developed by Landau and Lifshits. (Landau 1986) This solution describes deformations of the medium under the influence of a concentrated point force F applied on the surface. This relationship between displacement (u) and F can be represented using the Green's tensor G as

uᵢ=Gᵢⱼ(x,y,z)Fⱼ. (18)

We further assumed that all displacements are in-plane u=(uₓ u_{y})^{T}, that tractions normal to the displacement plane are zero F=(Fₓ F_{y})^{T} and that v is close to 0.5 for polyacrylamide hydrogels. (Schwarz 2002) The problem was therefore reduced the problem to x and y coordinates, (Butler 2002) which represent to the two dimensional movement of fluorescent microbeads being deformed due to cell tractions. Under these assumptions,

$${\tilde{G}}_{ij} = \frac{1 + υ}{π E} \frac{1}{{r}^{3}} [ \begin{matrix}( 1 - v ) {r}^{2} + {vx}^{2} & - vxy \\ - vxy & ( 1 - v ) {r}^{2} + {vy}^{2}\end{matrix} ] , \qquad ( 19 )$$

where r=\sqrt{x²+y²} and the off-diagonal elements were corrected with a minus sign. (Sabass 2008) To calculate the cell generated traction forces T(x,y), equation 17 can be represented as

uᵢ=∫∫Gᵢⱼ(x−x′,y−y′)Tⱼ(x′,y′)dx′dy′. (20)

Equation 19 corresponds to a spatial convolution of G and T, which Butler and colleagues first denoted as u=G⊗T, (Butler 2002) and represents displacement as a function of known tractions. To determine T as a function of u, we had to invert equation 20, which required transformation into the Fourier space because G is not diagonal. Using the convolution theorem, (Butler 2002) the problem becomes ũ(k)=\tilde{G}(k)\tilde{T}(k) and the transformed matrix \tilde{G} is expressed as

$${\tilde{G}}_{ij} = \frac{1 + υ}{π E} \frac{2 π}{{k}^{3}} [ \begin{matrix}( 1 - v ) {k}^{2} + {vk}_{y}^{2} & {vk}_{x} {k}_{y} \\ {vk}_{x} {k}_{y} & ( 1 - v ) {k}^{2} + {vk}_{x}^{2}\end{matrix} ] , \qquad ( 21 )$$

where k=\sqrt{kₓ²+k_{y}²} and kᵢ represent wave vectors.

We then computed traction forces through the inverse Fourier transformation,

T= [symbol] ⁻¹{\tilde{G}⁻¹ũ}. (22)

As detailed by Butler and colleagues, (Butler 2002) to solve this equation we solved the Nyquist frequency limitation by setting the off-diagonal elements of equation 20 to 0 if at a Nyquist frequency in x or y. We also filtered out the displacement values resultant from noise while calculating tractions as previously demonstrated by Schwarz and colleagues. (Schwarz 2002) In summary, to filter noise without altering signal, we achieved the smoothing with zero-order Tikhonov regularization, which was initially adapted by Sabass and colleagues (Sabass 2008) while solving this Fourier transformation problem. Sabass and colleagues altered Equation 21 into

T= [symbol] ⁻¹{(\tilde{G}^{T}\tilde{G}+λ²\tilde{H})⁻¹\tilde{G}^{T}ũ}. (23)

The regularization parameter λ determines the amount of the solution that originates from the regularization parameter relative to the data. H corresponds to the identity ∥₂ for a zero-order regularization.

In our MATLAB-based graphical user interface (reference), We implemented the possibility of presenting two traction force microscopy approaches to analyze our videos of moving microbeads: constrained and unconstrained traction force microscopy. These approaches were initially developed to quantify the forces of cell adhesion to deformable substrates. (Butler 2002) Butler and colleagues (Butler 2002) have shown that defining the deformed region of the gel is key for quantifying cell adhesion forces. To exclude erroneous solutions and the effect of noise, they implemented a constrained approach where generated forces are restricted to the area occupied by the cell. The opposite is an unconstrained approach where tractions outside of the area occupied by the cell are also considered. For the measurement of contractile forces generated by hiPSC-CMs, we implemented the option to do constrained or unconstrained Fourier-based traction force microscopy.

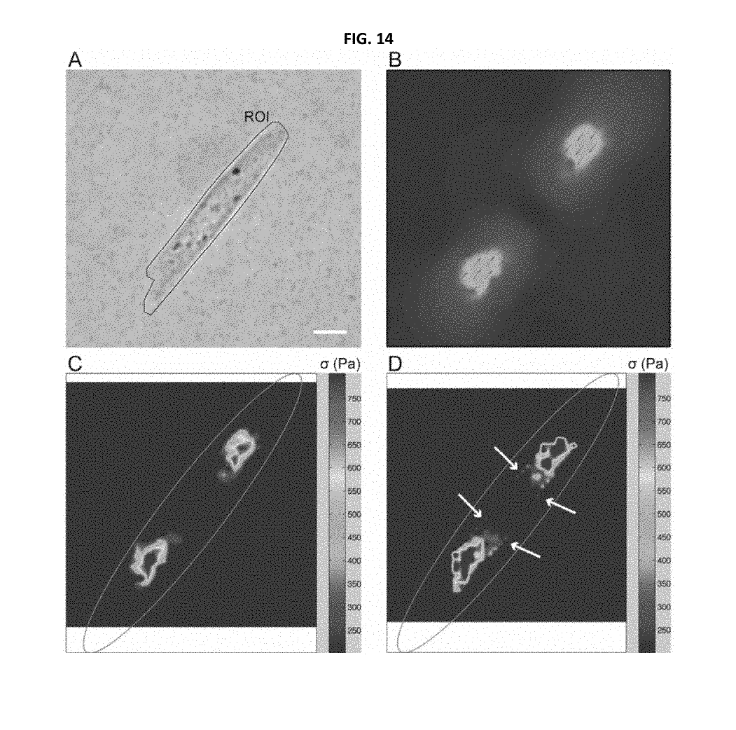

These methods generate maps of surface stresses (σ) that are converted to absolute values of traction forces (F) and we sum values of F (ΣF) by integrating all values of F over the respective areas where cells generate contractile forces. The constrained approach yields a map of cell-generated tractions within the ROI defined by the cell borders (FIG. 1B), while the unconstrained approach results from the direct conversion of force from displacement as above described. As shown in FIG. 14, constrained analysis computationally translates all tractions back to the area occupied by the cell, while with unconstrained analysis on can observe a more realistic translation of the contractile activity of hiPSC-CMs into tractions on the substrate. In the presented study, we used unconstrained traction force microscopy as now detailed.

Conversion of σ to F was done for each quadratic element of the traction grid that results from submitting videos of moving microbeads to traction force microscopy. We multiplied σ by the area of each respective grid element. For constrained force calculation, we integrated F within the ROI defined by the cell borders. For unconstrained measurements, we calculated an extended ellipse with the same center of mass as the ROI (FIG. 1E) and integrated F within the region delimited by this extended ellipse to determine ΣF. This approach has the advantages of allowing the analysis of one cell at a time within a video of multiple cells in an array and of not quantifying noise in regions of the substrate that are away from the cell. The area of the extended ellipse relates to the area of the ROI as follows:

Aₑₗₗᵢₚₛₑ=n·A_{ROI}. (24)

To calculate the extended ellipse, we set the constant n to values between 2 and 3 and set the orientation of the major axis (a) and of the minor axis (b) of the ellipse to respectively match the orientation of the major and minor axes of the ROI (FIG. 1E). Therefore,

(aₑₗₗ/=\sqrt{n}·a_{ROI}, (25)

and

bₑₗₗ=\sqrt{n}·b_{ROI}. (26)

We obtained plots of ΣF as a function of time from constrained and unconstrained Fourier-based traction force microscopy approaches (Butler 2002) applied to displacement maps of microbeads calculated from videos of moving microbeads. Displacement maps of microbeads were determined for each frame of a video relative to a reference frame.

As already detailed in the previous section, we selected a reference frame that corresponds to the relaxed state of a beating cell. For this effect, we used the same criteria defined by equations 12 and 13, but applied to videos of moving microbeads instead of brightfield videos of beating cells. The inputs for traction force microcopy analysis were displacement fields, hydrogel material stiffness E and Poisson ratio v (Equations 14, 16, 18 and 20). Our substrate had a Poisson ratio of 0.45 (Kandow 2007) and material stiffness of 10 kPa. (Ribeiro 2015)

Unconstrained Traction Force Microscopy

In the unconstrained analysis, we applied a Fourier transform to each displacement map. Then, for each wave number, we set tractions at f=0 to 0 (Equation 13) and computed G after equation 20 and set the diagonal elements to 0 at Nyquist frequency. (Butler 2002) We considered regularization as defined in Equation 23. We calculated the regularization parameter λ for the first frame of displacing microbeads in a video using the Regutools toolbox (MATLAB). (Hansen 2007a) Because noise does not vary within a video, we then applied the same parameter λ for the analysis of the subsequent frames. We calculated an independent value of λ for each analyzed video. After calculating stress values in the Fourier space for each pixel, we transformed stresses back to the real space and obtained a map of stresses for each frame.

A good calculation of λ is key to generate reliable solutions because Equation 23 represents an ill-posed problem, where arbitrarily small perturbations of input data can lead to an arbitrarily large perturbation of the solution. Calculation of λ via the Regutools toolbox (Hanson 2007a) solves a ill-posed problem defined as Ax=b that satisfies the following criteria:

a. the singular values of A tend to zero,

b. the ratio between the smallest non-zero values of A have large values.

A side constraint (Ω(x)) was introduced to minimize the norm ∥Ax−b∥, while minimizing Ωx). In the Tikhonov regularization approach, λ represents the weighing between the data and Ω(x)

minₓ∥Ax−b∥²+λ²Ω(x)². (27)

High values of originate an excessive level of smoothing, while small values increase the weight of noise pronounced in Ax=b. We also validated the ability of this approach to calculate a suitable λ with the L-curve criterion. (Hanson 2007b)

Constrained Traction Force Microscopy

In the constrained traction force microscopy analysis, (Dembo 1996) we required the same inputs that were used for the unconstrained analysis and also information on the ROI that limits the boundaries of the cell within the frames of moving microbeads. We first calculated stresses as detailed for the unconstrained calculation and defined a new traction field by setting the tractions outside of the ROI to zero. We then calculated the displacement field that corresponds to this new traction field and replaced experimental values of displacement inside the ROI by the calculated displacement values. (Dembo 1996) We iterated the calculation of stress from the displacements within the ROI to calculate new displacement values until we achieved stable values of stress within the ROI. The resultant stress values are then converted to force. However, the estimation of force with constrained traction force microscopy approach is very susceptible to noise because high noise leads to large force values at the cell boundary.

Sarcomere Length in Patterned hiPSC-CMs

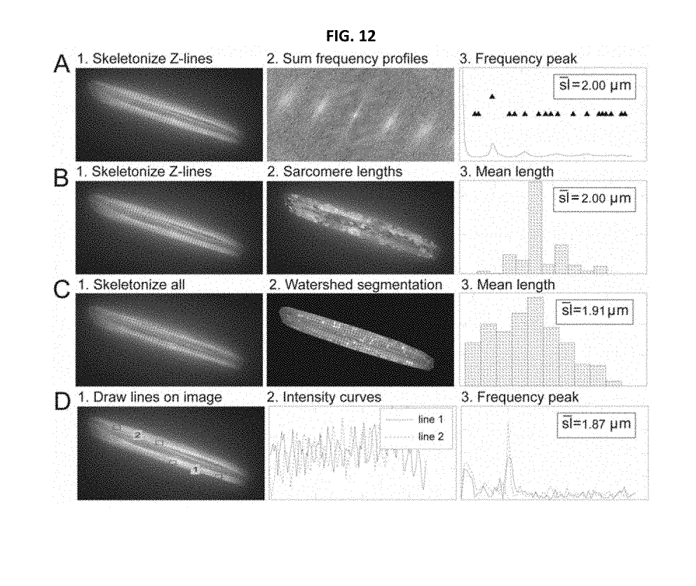

We analyzed videos of beating patterned hiPSC-CMs with LifeAct-labeled myofibrils (Ribeiro 2015a) to quantify the organization and dynamics of sarcomeres along myofibrils. Sarcomere shortening and movement during the contractile cycle was determined from analyzing how the size of all labeled sarcomeres in a single hiPSC-CM varies during each of its contractile cycles. The minimal length that separates two proximal Z-lines defines sarcomere size. LifeAct labels actin between Z-lines, which correspond to dark lines in LifeAct-labeled myofibrils. (Ribeiro 2015a) Therefore, the minimal distance between adjacent dark lines in LifeAct-labeled myofibrils defines sarcomere size. We used four different approaches to quantify sarcomere size along LifeAct-labeled myofibrils from frames of single beating hiPSC-CMs (FIG. 12) and used the second approach for performing better when analyzing videos with minimal user intervention.

First Approach

The first approach (FIG. 12A) is the current state of the art method to characterize sarcomere organization from immunocytochemically labeled sarcomeres, (Wang 2014) involves the skeletonization of LifeAct-labeled regions and was based on work initially developed by Kuo and colleagues. (Kuo 2012) As detailed by Hong and colleagues, (Lin 1998) we used a fingerprint enhancement algorithm available online (Kovesi 2000) to optimize the quality of the skeleton obtained from frames of Life-Act labeled myofibrils. The input of the algorithm was a set of frames of a video of moving labeled sarcomeres. For each frame, the algorithm identified ridge-like regions using the ridgesgment tool. Then ridge orientation was determined with the ridgeorient tool and sarcomeres were defined to be perpendicular to the orientation of adjacent myofibrils. Given this condition, the orientation map was rotated by π/2 and restricted to [0; π] because Z-lines are perpendicular relative to the orientation of myofibril direction and because it is irrelevant if the detected angle of myofibril orientation is α° or α-180°. Then, ridge frequencies across the image were determined with the ridgefreq tool and the ridgefilter tool enhanced the ridge pattern with signal filtering, originating a skeletonized image of myofibrils for each frame. All tools were downloaded from Peter's Functions for Computer Vision. (Kovesi 2000) After obtaining a skeletonized image of myofibrils, we determined an average sarcomere length using a radial Fourier transform and selecting the dominant frequency as described elsewhere. (Wang 2014, Kuo 2012) In summary, we summed the radial profiles of the Fourier-transformed skeletonized image to remove any user bias in selecting the main orientation of myofibrils and because we know a priori that the orientation of sarcomeres in patterned hiPSC-CMs is not strictly perpendicular to the cell's main axis. (Wang 2014) This summation of radial profiles leads to a one-dimensional curve (Γ(ω)), which is normalized to ensure that the integral over all frequencies equals 1. We then considered Γ (ω) to result from a combination of a periodic part (δₚ(ω)), which contains information on the periodicity of sarcomeres, with an aperiodic part (δ_{AP}(ω)) describing artifacts from imperfect skeletonization,

$$\begin{matrix}{Γ}_{P} ( ω ) = {\sum }_{k = 1}^{5} {a}_{k} {e}^{[ - {( \frac{ω - k {ω}_{0}}{{δ}_{k}} )}^{2} ]} , & ( 28 ) \\ {Γ}_{AP} ( ω ) = a + {be}^{( - c ω )} , & ( 29 )\end{matrix}$$

where Γ_{P} was approximated by a series of 5 Gaussian peaks, which occur at the mean sarcomere frequency. Least-square fitting was then applied to estimate the parameters a, b, c, ω_{θ}, aₖ and δₖ. The area under Γ_{P} was also registered as a measure of sarcomere organization. A higher area under the major frequency component indicates that sarcomeres are more periodically organized. With this approach, the mean Z-line frequency (r₀) was determined by the frequency parameter (ω₀=1/r₀). In detail, the algorithm that calculated the main frequency from orientation-averaged Fourier transforms used a sarcomere skeleton as input and the output was the average sarcomere length. Each rectangular frame was transformed into a square image by adding zero values to the shorter side of the rectangular frame until each side had the same size. The resultant square image was then divided into n angles. The skeleton image was rotated for each angle and 1D-Fourier transform was submitted along the x direction for each angle of image rotation. The Fourier profiles of each angle were then summed. The radial amplitude defined by Γ_{P} was summed with the inspect tool (Kovesi 2000) and Γ_{P} was normalized to yield a curve with a total area of 1. We considered sarcomere lengths in the 2-μm range. The maximum peak of Γₚ was determined within this sarcomere range by first fitting a p-th order polynomial to Γ_{P} and guessing the frequency peak ω₀ as the maximum point of Γ_{P} within a frequency range of [0.7. ω₀; 1.3. ω₀]. The mean sarcomere length was then computed as the inverse of the dominant frequency peak r₀.

Second Approach

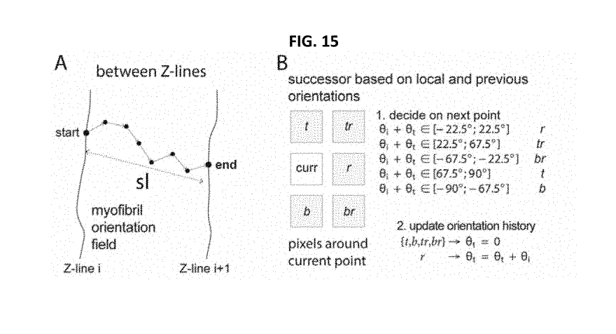

The second approach (FIG. 12B) is a novel method that we developed to determine sarcomere lengths in each frame without the need of curve-fitting procedures or radial Fourier transforms. The approach consists of automatically measuring the length of the segment between adjacent Z-lines that is parallel to the direction of myofibril alignment (FIG. 15). The algorithm for this approach measured the length from Z-line i to Z-line i+1 using information on myofibril orientation in the region around Z-line i and Z-line i+1 and considering a skeleton of sarcomeres generated as previously detailed for the first approach. The algorithm developed first a map of all points that compose the sarcomere skeleton and each point was taken once as a starting location of a path along the direction of myofibril orientation that stopped when another Z-line in the skeleton was reached (FIG. 15). The length between adjacent Z-lines was therefore defined as the Euclidean distance between start and end points calculated with this method (FIG. 15). The path was calculated pixel by pixel within the skeletonized image. For each new pixel of the path, the algorithm evaluated what the other pixel of the path was based on the local orientation of the myofibril. For pixels that were starting points or pixels already in a path, the local orientation angle of myofibrils was taken as the deciding factor to determine the next path pixel. In the algorithm, we particularly defined that the y direction of the path could be chosen freely, but only pixels in the +x direction could be candidates for the next element of the path (FIG. 15). This definition derived from the fact that every point composing the skeleton was considered as a starting point and because orientations of myofibrils are in the [0,Th] range. In relation to a known point of a path, the next neighboring pixel to be included as the next element of that path could be the pixel on the right, on the top, on the bottom, on the top right or on the bottom right (FIG. 15-B). Given this condition, a 10° angle or a 0° angle of known myofibril orientation lead to the same decision for the next pixel to include in the path: the pixel on the right. We estimated the local orientation of myofibrils through the MATLAB-written code RIDGEORIENT. (Kovesi 2000) This tool indicates the principle ridge direction through local gradient variations. By definition, the gradient is tangential to the main orientation. (Krause 2009)

The global orientation of a myofibril between Z-lines was also taken in consideration for deciding the next pixel in the path between Z-lines (FIG. 15) because small angles can add up during the extending of the line defined by the path and better reveal the real myofibril orientation. For example, starting at a pixel on a Z-line (x₁, y₁) with a local orientation angle θ₁=10°, the next pixel would have to be (x₁+1, y₁). If θ₂=10°, the next pixel would again be to the right, adding (x₁+2, y₁) to the path. If θ₃=15° and the orientation angles of the previous path elements are ignored, the next pixel to be included in the path should be (x₁ 3, y₁). However, if the angles of local orientation of all path elements added up to θₜ=Σᵢ³θᵢ=35°, the correct solution would be to add the pixel on the top right side of the last known path element (x₁+3, y₁+1). If θ₄=10° after this effect of adding orientation angles, then the next pixel to be added to the path would be (x₁+4, y₁+1). FIG. 15 illustrates how this algorithm worked for choosing the path that determined the distance between Z-lines. A maximal iteration number was set for determining the path between Z-lines to exclude faulty measurements due to holes in the skeleton or incoherent orientation maps. In summary, to obtain an output of sarcomere size from an input of skeletonized frames, all points of the skeleton were considered for the beginning of a path of the segment that separates Z-lines. Then, while the path was not outside of the image window and was shorter than the set maximal limits of sarcomere length and iteration number, the sum of local orientation angle and angle history θᵢ+θₜ were computed. Based on the obtained orientation values, the next pixel of each path was chosen until the path reaches a Z-line and all requirements are satisfied. Once the path was determined, the Euclidian distance between start and end of the path corresponded to the sarcomere length, which was also related to the sarcomere orientation angle.

Third Approach

We developed another novel approach to quantity the dimensions of sarcomeres (FIG. 12-C). However, this approach performed poorly compared to the first and second approaches. We now detail our efforts and rationale for the development of this approach (FIG. 12-C). We used gradient watersheds for segmenting sarcomeres in an image and fit a rectangle to the region occupied by fluorescently labeled actin between Z-lines. The goal was to identify sarcomeres in the real space and their dimensions. We termed this rectangle sarcomere box and its sides fit the region occupied by sarcomeres between Z-lines. In summary, to delineate the space occupied by each box, we initially submitted the images of myofibrils to two skeletonization steps: i skeletonization of actin between Z-lines that reveals structures aligned in the direction of myofibrils and ii) skeletonization of Z-lines that results in images of structures aligned in a direction perpendicular to myofibril alignment. We then combined both skeletons to generate a grid composed of sarcomere boxes and fitted an ellipse to each box to determine the orientation of each sarcomere within the cell. We use the information on sarcomere orientation and the dimensions of the sarcomere box to calculate sarcomere length.

We now describe in detail the different steps used in this approach. We transformed gray-scale frames of LifeAct-labeled myofibrils into three-dimensional topographical maps, in which the grey-scale intensity value of each pixel represents an altitude value. Our use of the gradient map in this approach was based on the fact that intensity gradients are high at the borders of Z-lines. The frames were binarized to separate and identify Z-lines, before watershed segmentation was applied to them. For this process, we also used the ridge-enhancing algorithm, already detailed in the description of approach 1, because it considerably improved the quality of the segmentation. This step is especially necessary for frames with inconsistent or uneven fluorescent distribution and for sequences of frames where intensity values are time-dependent due to the effects of photo-bleaching. After segmentation of sarcomeres, myofibril orientation and local frequencies were estimated with Fourier analysis as also already detailed for the first approach. Then, we used the knowledge that the orientation of Z-lines is perpendicular to the orientation of myofibrils to apply a second type of ridge enhancing routine. For this approach, a sarcomere map is obtained by combining both of these routines. We fitted a sarcomere box to the space between detected Z-lines by using information on the orientation of myofibrils. For this purpose, we first fitted an ellipse to the region occupied by each sarcomere, which we geometrically characterized with a major and minor axis, as well as with an orientation for each of the two axes. These axes coincide with two different levels of sarcomere orientation: Z-line orientation and myofibril orientation. Within a sarcomere, actin has an orientation perpendicular to Z-lines and these orientations can match the orientation of the main axis or major axis of the sarcomere box. We computed the length of sarcomeres by fitting a rectangle to the sarcomere space between detected Z-lines and oriented in the direction of myofibril alignment. We then fitted an ellipse to this rectangle by using its geometrical definition to match the dimensions of the box that delimits the sarcomere space.

The orientation of myofibrils was rotated by π/2 if the orientation of the main axis of the fitted ellipses was along the direction of Z-lines. We defined the ellipse in cylindrical coordinates to facilitate this task,

$$r ( α ) = \frac{a \cdot b}{\sqrt{{( b cos α )}^{2} + {( a sin α )}^{2}}} , \qquad ( 30 )$$

where α is the angle around the center of the ellipse, r is the distance between the center and the ellipse line, a is the major axis and b is the minor axis. After calculating the correct orientation of the sarcomere region, we assured that the dimensions of the sarcomere box were correct by using the criterion of area correspondence between the original segment and the box fit. The correct rectangular dimensions of sarcomere boxes were found by requesting area correspondence between the fitted ellipse and the rectangle with the sarcomere dimensions to be determined. A correction factor c assured the correct geometrical shape,

$$c = \frac{{A}_{1}}{{A}_{2}} = \frac{π}{4} . \qquad ( 31 )$$

The correct sarcomere dimensions w_{new} and h_{new} were obtained by demanding equal area between the rectangle that fits the sarcomere with area A₂ and the initial ellipse with area A₁. The correction factor c was introduced to compensate for the fact that A₁ corresponds to the area of an ellipse. Claiming equal area yields

$${h}_{new} \cdot {w}_{new} = {A}_{2} ⇔ \frac{h}{{h}_{new}} = \frac{w}{{w}_{new}} . \qquad ( 32 )$$

Given these conditions, dimensions for each sarcomere box were then calculated as follows

$${h}_{new} = \sqrt{{A}_{2} \cdot \frac{h}{w} \cdot c} ; {w}_{new} = \sqrt{{A}_{2} \cdot \frac{w}{h} \cdot c} . \qquad ( 33 )$$

We now summarize the algorithm that we developed for this approach. The input consisted of frames of labeled sarcomeres and maximal and minimal values of sizes that sarcomere can have. To obtain an output of sarcomere length distribution, we first performed Z-line skeletonization and repeated the skeletonization routine to delineate actin bundles between Z-lines. We combined both skeletons resultant from the previous steps into a new skeleton and closed small holes to perform the watershed transform. We finally calculated the rectangular dimensions of each individual sarcomere by fitting an ellipse. The orientation angle of the ellipse and the values of its major and minor axis were used to finally determine sarcomere dimensions. We discarded sarcomeres with calculated lengths that did not match the range of known sarcomere lengths. The sarcomere rectangle was rotated to the correct orientation and the value of the calculated length was added to the distribution vector for each sarcomere (FIG. 12-C).

Fourth Approach

To our knowledge, the last tested approach (FIG. 12-D) was developed by Bray and colleagues (Bray 2008) and consists of drawing line scans along myofibrils, plotting the intensity profile along those lines and measuring the length between the bands that correspond to Z-lines.

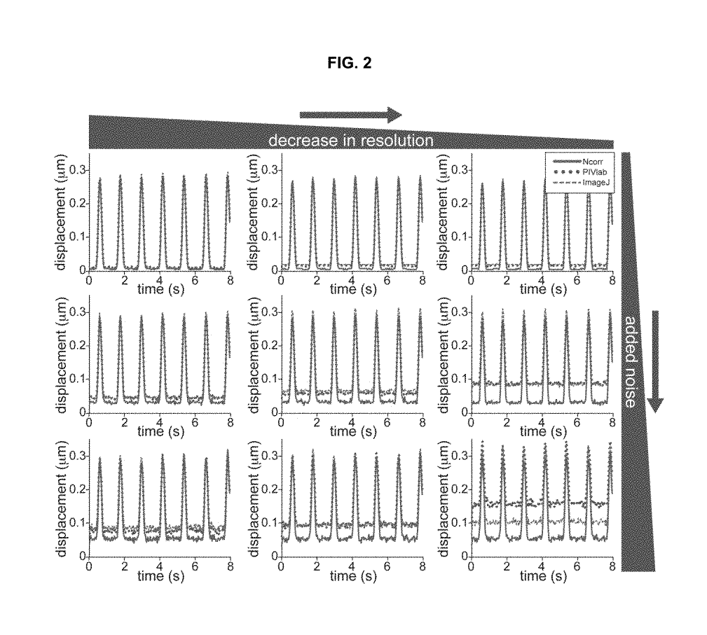

The second approach performed better for analyzing sarcomere lengths in videos of LifeAct labeled beating cells. This approach was applied to different frames in a video to determine how the different properties measure with traction force microscopy or from cell movement related to sarcomere size, movement and orientation during the contractile cycle. Most of the tested approaches relied on successful skeletonization of each frame, which may not be perfect due to noise or inconsistent illumination. To quantitatively compare different skeletons from different frames in a video, we generated a master skeleton based on the skeletons generated from all frames. We obtained N−1 pseudo skeletons and one reference skeleton from all video frames i=1, 2, 3, . . . , N. Then we used a threshold to determine the certainty that a pixel is part of the master skeleton. For example, a threshold of 0.6 leads to a master skeleton with ridges where 60% of pseudo-skeletons from all frames showed ridges. For processing each video, we first skeletonized each frame into ridges and chose a frame to be used as a reference and calculate the displacement of sarcomeres during the contractions. We used this displacement information to calculate a pseudo-reference skeleton for each frame. Then, we integrated pseudo-reference skeletons into the master skeleton and set a threshold for confidence to finally calculate the final skeletons for each frame i using the same displacement results. Displacements were calculated with the cross-correlation algorithm Ncorr, (Blaber 2015) because it is a cross-correlation approach with high performance in characterizing movement (FIG. 2). From this analysis, we also obtained all parameters associated to movement that we also obtained from videos of cells imaged with brightfield and previously detailed.

### II. RESULTS

A. The Mechanical Output of μPatterned hiPSC-CMs is Quantified from Microscopy Videos

We acquired videos of live single beating μpatterned hiPSC-CMs on polyacrylamide hydrogels to analyze the mechanical output of their contractile cycle (FIG. 1A). We acquired three different types of videos with microscopy: brightfield videos of single cells (Online Movie I), green fluorescent videos of microbeads dispersed in the substrate (Online Movie II) and red fluorescent videos of labeled myofibrils (Online Movie III). Movement of ρbeads (Online Movie II) occurs due to cell traction stresses (σ) exerted on the substrate and to cell stable adhesions to the gel surface. (Ribeiro 2015a) We transfected cells with LifeAct to fluorescently decorate myofibrils and detect sarcomere Z-lines (see Methods). (Ribeiro 2015a) Each video shows the movement of different structures: cell surface, substrate and myofibrils. The different types of movement are related to each other because their driving force results from the contractile activity of sarcomeres.

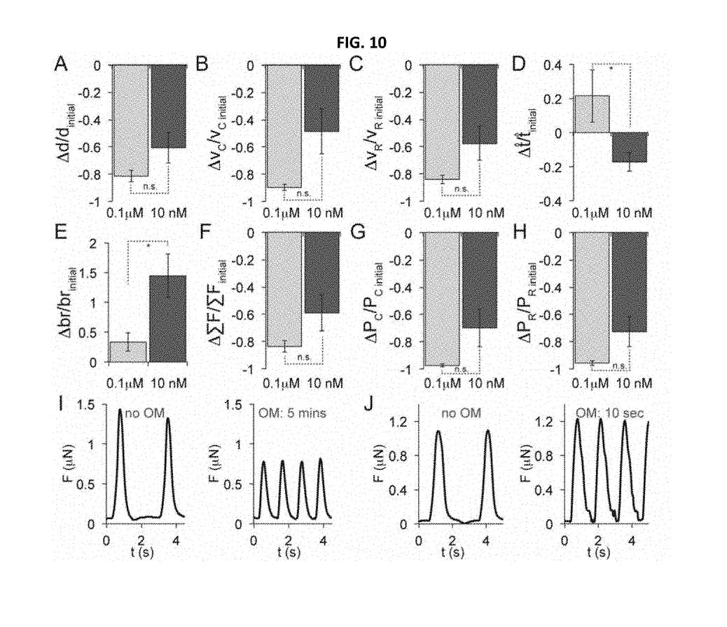

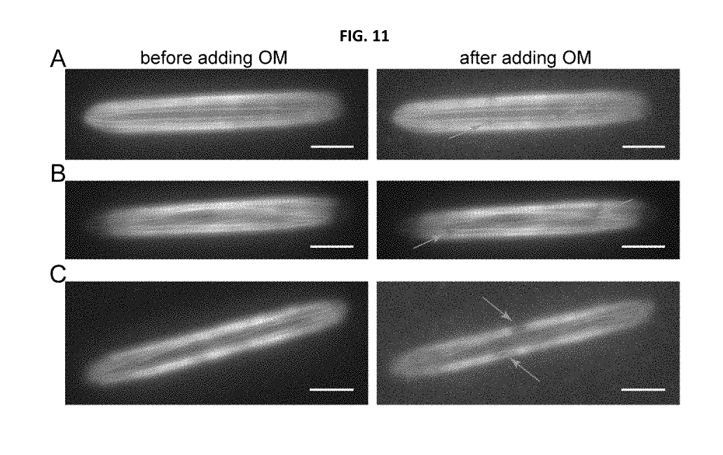

We aimed to develop an integrated tool that analyzes these videos and generates different parameters that evaluate different functional facets of the contractility and kinetics of beating μpatterned hiPSC-CMs. To achieve these aims, we first tested approaches to determine curves of average displacement (d-curves) and curves of average velocity of displacement (V-curves) from the different types of videos (FIG. 1B-L). For a region of interest (ROI) defined by the borders of each μpatterned hiPSC-CM (FIG. 1B) in brightfield videos, we calculated d (FIG. 10) and V (FIG. 1D) with the cross-correlation algorithm Ncorr (Blaber 2015) as detailed in the Methods section. We also used Ncorr to determine the movement of μbeads (FIG. 1D) within a substrate surface region delimited by an ellipse of dimensions that are proportional to the area and shape of the ROI (see Methods) and also obtained d-curves (FIG. 1F) and V-curves (FIG. 1G) of moving μbeads. After obtaining the map of μbead displacement for each video frame, we estimated σ with traction force microscopy for each video frame (see Methods) and summed the absolute values of the forces (ΣF) corresponding to each σ value (see Methods) to obtain F-curves. By multiplying ΣF by V, we estimated the contractile power output (P) and calculated P-curves (FIG. 11) of μpatterned hiPSC-CMs. We also used Ncorr to characterize the movement of videos of moving myofibrils in single cells (FIG. 1J) to determine d-curves (FIG. 1K) and V-curves (FIG. 1L). The curves presented in FIG. 1 are the basis for analyzing the contractile mechanical output of single μpatterned hiPSC-CM and provide information on the contractile performance of these cells. However, calculating the curves presented in FIG. 1 relies on the ability of Ncorr to systematically analyze movement with high precision.

To test if Ncorr was a suitable approach for quantifying the contractile displacement of μpatterned hiPSC-CMs, we compared Ncorr with two other cross-correlation algorithms that have been previously used to analyze movement at the micron scale: PIVIab, and ImageJ PIV. (Tseng 2012) We processed the ROI defined by the cell borders in Online Movie IV with Ncorr, PIVIab and ImageJ PIV and obtained similar d-curves (FIG. 2). We then decreased image resolution and added noise to the frames of Online Movie IV to test if the different cross-correlation approaches yielded similar results independently of the video image quality. Ncorr demonstrated a better performance in systematically yielding the same displacements from videos with varying image quality. For all the analyses illustrated in FIG. 1, where systematic performance in processing videos with low-to-medium noise and variable resolutions is required, Ncorr seemed to provide more consistent results.

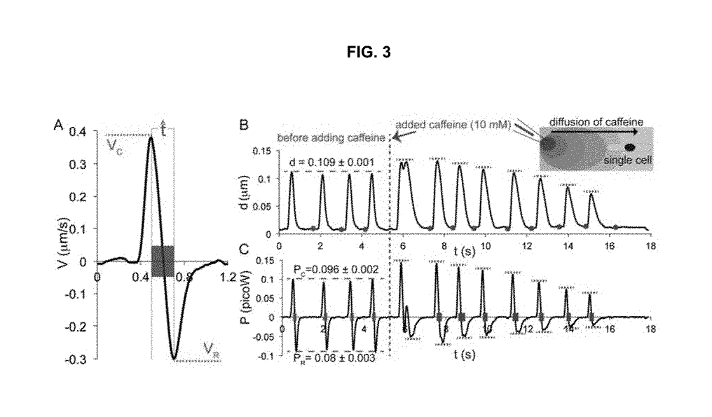

Overall, we calculated two types of parameters to describe the mechanical output of μpatterned hiPSC-CMs from the curves presented in FIG. 1: contractile parameters and kinetic parameters. Contractile parameters, such as d and ΣF relate to the amount of stresses that each cell can generate during their contractile cycle. Beat rate (br) and V are kinetic parameters. BR describes the time between contractile cycles and V represents the velocity of contraction or relaxation. P is a parameter that provides both contractile and kinetic information because it is calculated from ΣF and V. We specifically determined peak displacement (dₘₐₓ), peak force (ΣFₘₐₓ), peak velocity of contraction (V_{C}), peak velocity of relaxation (V_{R}), peak power of contraction (P_{C}) and peak power of relaxation (P_{R}). We also calculated the time between peak velocity of contraction and peak velocity of relaxation (i) (FIG. 3A). This kinetic parameter scales with the total time of contraction and can also be simply determined from V-curves or P-curves. For example, we observed an increase in \hat{t} after exposing the cell to low doses of caffeine by slowly diffusing it through the cell culture media (FIG. 3B). This observation clearly illustrated how \hat{t} scales with the time of each contractile cycle and suggested that our approaches to evaluate the contractility of hiPSC-CMs can detect the effects of drugs that affect cardiac activity. We also observed caffeine-induced variations in dₘₐₓ, P_{C} and P_{R} (FIGS. 3B and C). We therefore further tested the ability of our combined analysis to detect and quantify drug-induced changes in cell contractility.

B. Detection of Drug-Induced Changes in the Contractility of μPatterned hiPSC-CMs

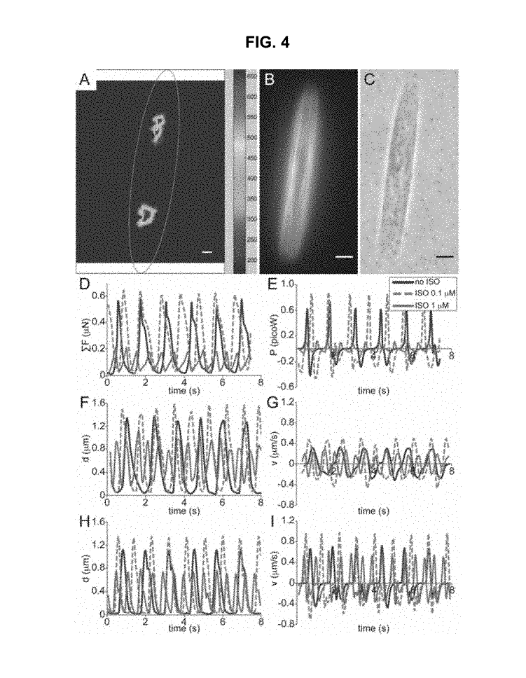

Specific drugs or small molecules can change the contractile activity of hiPSC-CMs by affecting pathways or proteins that regulate heart beating and function. (Butler 2015) We incubated μpatterned hiPSC-CMs in isoproterenol at concentrations of 0.1 μM and 1 μM and analyzed variations in their mechanical output as defined in the previous section. Isoproterenol activates the β-adrenergic pathway and has different effects on the contractility of CMs in a dose dependent manner. (Katano 1992) Isoproterenol has been reported to induce positive inotropic and positive chronotropic responses in CMs at 0.1 μM, (Butler 2015) which respectively corresponds to an increase in contractile mechanical output and increase in beat rate. In hiPSC-CMs in 1 μM isoproterenol, mechanical output has been shown to decrease (acting as a negative inotrope), while beat rate has been shown to increase (acting as a positive chronotrope). (Yokoo 2009) We observed similar responses to 0.1 μM and 1 μM isoproterenol in μpatterned hiPSC-CMs (FIG. 4). For the same single cell, we simultaneously measured contractile-induced surface stresses with traction force microscopy (FIG. 4A), movement of myofibrils (FIG. 4B) and the movement of the cell imaged with brightfield (FIG. 4C). Both ΣFₘₐₓ and br increased when isoproterenol was added to the media at a concentration of 0.1 μM (FIG. 4D), as well as P_{C} and P_{R} (FIG. 4E). As expected, except for a clear increase in br, 1 μM of isoproterenol induced a substantial decrease in all these parameters. Curves obtained from processing videos of moving fluorescent myofibrils (FIGS. 4F and 4G, Online Movies V,VI and VII) and moving cells imaged with brightfield (FIGS. 4H and 41) showed a similar trend. Contractile and kinetic parameters obtained from analyzing myofibril and cell d-curves and V-curves presented the same levels of variation as observed for the obtained F-curve and P-curve. In these analyses, d was a proxy to ΣF and V was a proxy to P. Increase in dₘₐₓ, f, V_{C} and V_{R} was detected when isoproterenol was added at a concentration of 0.1 μM. A more pronounced increase in f was observed after adding isoproterenol at a concentration of 1 μM, but the absolute values of dₘₐₓ, V_{C} and V_{R} decreased. However, the variations in ΣFₘₐₓ, P_{C} and P_{R} measured with traction force microscopy (FIGS. 4D and 4E) after adding isoproterenol were more pronounced than the variations in dₘₐₓ, V_{C} and V_{R} measured from videos of moving myofibrils (FIGS. 4F and 4G) or of a beating cell (FIGS. 4H and 41). This observation suggests that results of traction force microscopy do not necessarily match the movement of myofibrils or the movement of cells. In addition, no notable differences were qualitatively observed between the levels of variation in dₘₐₓ, V_{C} and V_{R} measured either from myofibril movement or from cell movement (FIGS. 4F-I).

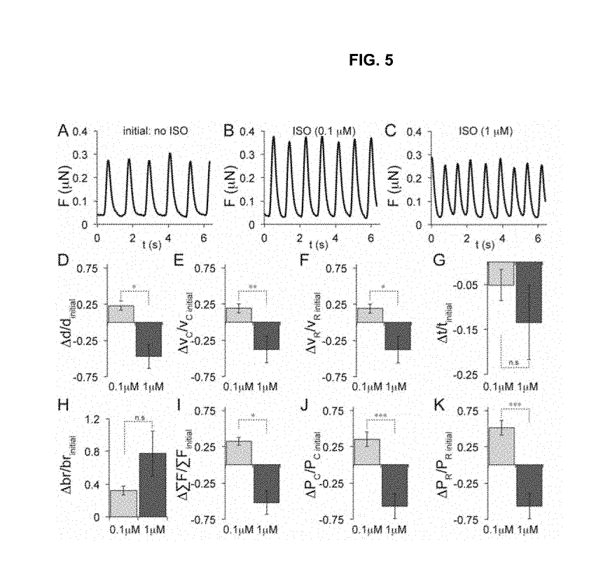

We then measured contractile variations in six single μpatterned hiPSC-CMs after being incubated first in 0.1 μM and then in 1 μM of isoproterenol (FIG. 5) to further test the ability of this approach to consistently assay populations of cells. Myofibrils in these cells were not labeled with LifeAct. Therefore we only analyzed brightfield videos and fluorescent videos of moving myofibrils for these cells. We acquired videos for each concentration of isoproterenol added to the cell medium. In general for these cells, we also observed an increase in ΣFₘₐₓ and br for 0.1 μM and a decrease in ΣFₘₐₓ followed by a more pronounced increase in br for 1 μM (FIGS. 5A-C). We then analyzed variations in all contractile parameters that we could evaluate from traction force microscopy (FIGS. 5D-K). For any measured parameter x of mechanical output, we measured variation as

Δx/xᵢₙᵢₜᵢₐₗ=(x(ISO)−xᵢₙᵢₜᵢₐₗ)/xᵢₙᵢₜᵢₐₗ. (1)

We observed different variations in the following contractile parameters obtained from traction force microscopy between the effects of 0.1 μM and 1 μM in cell mechanical output: dₘₐₓ, V_{C}, V_{R}, ΣFₘₐₓ, P_{C} and P_{R} (FIGS. 5D-F and 5I-K). The absolute values of these parameters for each cell consistently increased for 0.1 μM isoproterenol and decreased for 1 μM isoproterenol. Values of \hat{t} decreased (FIG. 5G) and values of br increased (FIG. 5 H) with isoproterenol, but no statistically significant differences were detected in the variations of these specific parameters when cells were exposed to the two concentrations of isoproterenol. These results demonstrated the ability of the presented traction force microscopy analytical tool to consistently analyze drug-induced changes in the contractility of populations of μpatterned hiPSC-CMs.

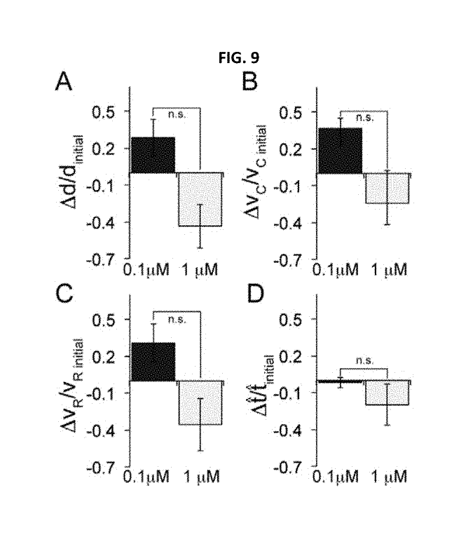

We generally observed a similar trend in the variations of parameters (d, V_{C}, V_{R}, \hat{t}) calculated from the analysis of displacements within ROIs in brightfield videos of cells incubated in different concentrations of isoproterenol (FIG. 9). However, analyzing brightfield videos did not yield differences in variations of parameters with statistical significance (FIG. 9). This result may suggest that a higher number of brightfield videos of cells must be analyzed to achieve differences with statistical significance between parameters of mechanical output when cells are in different concentrations of isoproterenol.

The contractile and kinetic effects of isoproterenol in CMs are well understood and characterized (Butler 2015) and they were detected in μpatterned hiPSC-CMs with our tools for analyzing cell mechanical output. However, the contractile and kinetic effects of isoproterenol in CMs are downstream of β-adrenergic signaling activation and do not result from direct alterations in specific myofilament proteins. To test the detection of contractile variations due to changes in the binding of myosin to thin filaments in μpatterned hiPSC-CMs, we incubated cells in omecamtiv mecarbil. Omecamtiv mecarbil is a cardiac-specific myosin activator that accelerates the transition of myosin binding to actin towards a strongly bound state (Kuo 2012). We tested the effects of 0.1 μM and 10 nM of omecamtiv mecarbil in the mechanical output of μpatterned hiPSC-CMs and calculated variations in parameters derived from traction force microscopy (FIG. 10 A-H). We acquired videos for this analysis (FIG. 1A) within 5 minutes after adding omecamtiv mecarbil to the cell culture medium. Variations of \hat{t} and br were statistically different between cells incubated in 0.1 μM and 10 nM of omecamtiv mecarbil. In summary, we observed decreased mechanical output (negative inotropy) of μpatterned hiPSC-CMs induced by omecamtiv mecarbil and chronotropic effects on cell contractility depended on the dose of omecamtiv mecarbil (FIG. 10-I).

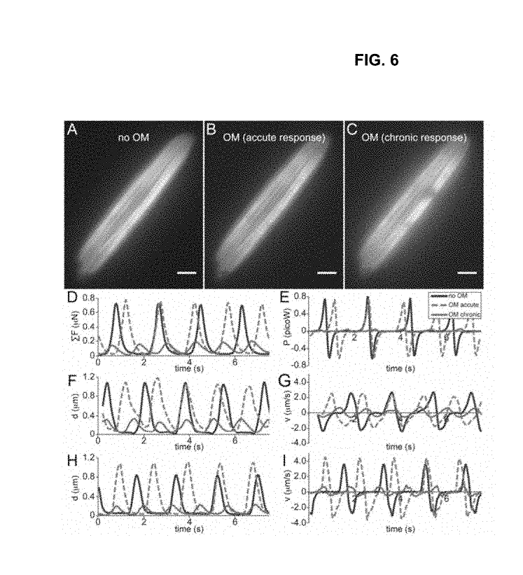

We then tested the instantaneous acute effects of omecamtiv mecarbil in the contractility of a single cell within the initial seconds of incubation (FIG. 10J). In opposition to the chronic effects detected within 5 minutes of adding omecamtiv mecarbil, we observed positive inotropy in this single μpatterned hiPSC-CM within 10 seconds of adding 0.1 μM of omecamtiv mecarbil. We further investigated these differences in acute and chronic contractile effects with a single μpatterned hiPSC-CM with fluorescently labeled myofibrils (FIG. 6A and Online Movie VIII). We aimed to know, for this small molecule, if parameters obtained from traction force microscopy related with parameters obtained from analyzing videos of moving myofibrils and brightfield videos of beating cells.

The acute response of a single hiPSC-CM to omecamtiv mecarbil was characterized by changes in sarcomere organization (FIG. 6B) and movement (Online Movie IX). For each contractile cycle, we observed oscillatory contractions of sarcomeres and overlap between sarcomeres (Online Movie IX). We then detected chronic effects of omecamtiv mecarbil on the organization of myofibrils. These effects consisted of myofibril damaging (FIG. 6C and Online Movie X). Such level of damage was also observed when μpatterned hiPSC-CMs were incubated in 1 μM and 10 nM (FIG. 11). For the cell presented in FIG. 6, we also analyzed its mechanical output with traction force microscopy (FIGS. 6D and 6E), analyzed the movement of myofibrils (FIGS. 6F and 6G) and analyzed the movement of the cell imaged with brightfield (FIG. 6H an 61). As also shown in FIG. 10 J, we observed a slight acute increase in ΣFₘₐₓ and in br for this cell (FIG. 6D). However, the absolute values of P_{C} and P_{R} did not vary right after adding omecamtiv mecarbil and even decreased in some contractile events.

Analysis of myofibril movement yielded similar variations of parameters of mechanical output. The acute values of dₘₐₓ and br slightly increased (FIG. 6F), but no considerable acute variations were observed in V_{C} and V_{R} (FIG. 6G). In opposition to what we observed with isoproterenol (FIG. 4), with omecamtiv mecarbil the analysis of cell movement from brightfield videos originated different results from the analysis of movement of myofibrils. From brightfield videos, we detected a considerable acute increase in dₘₐₓ (FIG. 6H) and an increase in the absolute values of V_{C} and V_{R} (FIG. 6I).

Overall for variations in mechanical output induced by omecamtiv mecarbil, parameters coincided between analyzing traction force microscopy results and myofibril movement, but differed from changes in cell movement imaged with brightfield. In opposition, the detection of variations induced by isoproterenol yielded a similar trend between the different analytical approaches (FIG. 4). Quantified parameters of mechanical output derived from the different curves are presented in Table I for the cell exposed to isoproterenol (FIG. 4) and the cell exposed to omecamtiv mecarbil (FIG. 6).

TABLE 1
ISO

| | | ΣFₘₐₓ (μN) | V_{R} (μm/s) | V_{C} (μm/s) | P_{R} (picoW) | P_{C} (picoW) |
|---|---|---|---|---|---|---|
| TFM | no ISO | 0.53 | 1.05 | 1.68 | 0.28 | 0.7 |
| | 0.1 | 0.58 | 0.85 | 1.92 | 0.36 | 0.86 |
| | 1 | 0.15 | 0.93 | 0.43 | 0.06 | 0.1 |
| | | d (μm) | V_{R} (μm/s) | V_{C} (μm/s) | a_{θ} (°) | a_{δ} (s) |
| LifeAct | no ISO | 1.22 | 2.54 | 2.63 | 55.4 | 0.1 |
| | 0.1 | 1.43 | 3.45 | 4.78 | 19.5 | 0.31 |
| | 1 | 0.71 | 2.05 | 2.02 | 20.8 | 0.28 |
| brightfield | no ISO | 1.09 | 3.78 | 6.85 | 31.4 | 0.01 |
| | 0.1 | 1.3 | 5.37 | 9.3 | 75.5 | 0.02 |
| | 1 | 0.68 | 3.65 | 5.34 | 25.3 | 0.02 |

OM

| | | ΣFₘₐₓ (μN) | V_{R} (μm/s) | V_{C} (μm/s) | P_{R} (picoW) | P_{C} (picoW) |
|---|---|---|---|---|---|---|
| TFM | no OM | 0.7 | 1.18 | 1.35 | 0.6 | 0.75 |
| | chronic | 0.72 | 1.1 | 1.22 | 0.59 | 0.67 |
| | acute | 0.14 | 0.18 | 0.29 | 0.02 | 0.04 |
| | | d (μm) | V_{R} (μm/s) | V_{C} (μm/s) | a_{θ} (°) | a_{δ} (s) |
| LifeAct | no OM | 1.03 | 2.2 | 2.25 | 36.3 | 0.05 |
| | chronic | 0.93 | 1.63 | 2.2 | 17.1 | 0.88 |
| | acute | 0.25 | 0.51 | 0.54 | 40.9 | 0.89 |
| brightfield | no OM | 0.79 | 2.87 | 3.53 | 44.5 | 0.03 |
| | chronic | 1.05 | 3.42 | 4.35 | 68.8 | 0.04 |
| | acute | 0.18 | 0.54 | 0.71 | 70.1 | 0.21 |

### C. Detection of Variations in Sarcomere Length Related to Changes in Mechanical Output

Labeling myofibrils in live hiPSC-CMs allows the quantification of sarcomere length (sl) and therefore the quantification of sarcomere shortening during the contractile cycle of cells. (Ribeiro 2015a) We developed an automated tool to quantify sl for each frame of a video of moving μpatterned hiPSC-CMs with fluorescently labeled myofibrils. Developing this tool involved testing four different approaches to measure sl from video frames (FIG. 12). The detailed steps involved in each approach are described in the Methods section. In general, the first three approaches consisted of a sequence of automated image processing steps that followed the skeletonization (Kuo 2012) of sarcomeres. In the first approach (FIG. 12-A), each frame of skeletonized sarcomeres was submitted to Fourier analysis and sl was calculated from the dominant peak of the sum formed by the radial Fourier transforms of the captured images. (Wang 2014) The second approach (FIG. 12-B) consisted of automatically calculating the distance between Z-lines in the skeletonized frame taking into consideration the orientation of myofibrils. Watershed segmentation was used in the third approach to isolate each single sarcomere from the skeletonized frame. The fourth approach consisted of analyzing line scans of fluorescently labeled myofibrils drawn along the direction of myofibril alignment. (Ribeiro 2015a) We calculated average sl values from a frame of a cell with labeled myofibrils with each of these approaches (FIG. 12). The first and second approaches coincided in the value of average sl and showed low variability in sl values within sarcomeres. The high variability in sl values obtained with the third and fourth approaches made them less appropriate for analyzing sarcomeres. The second approach yields information on different sl values within the cell, while the first approach only provides information on the dominant sl value. In addition, selecting the dominant peak (FIG. 12-A) is not a trivial task to automate. Therefore, we used the second approach in our analytical tool set to calculate sl.

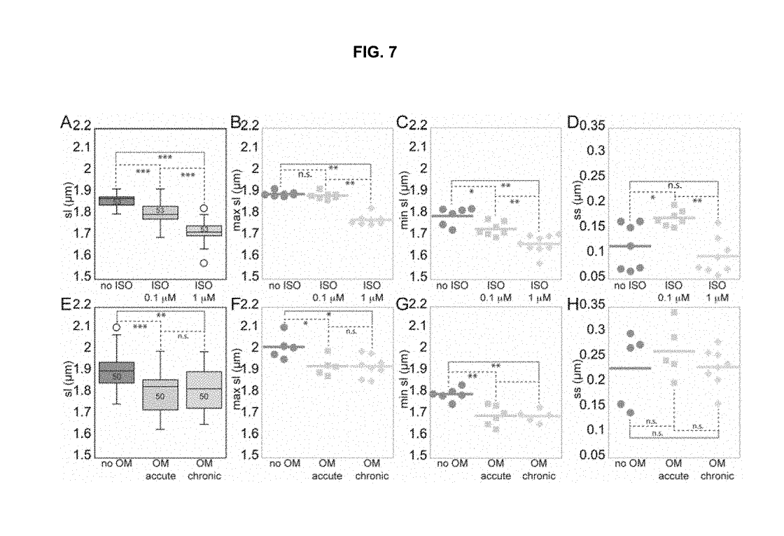

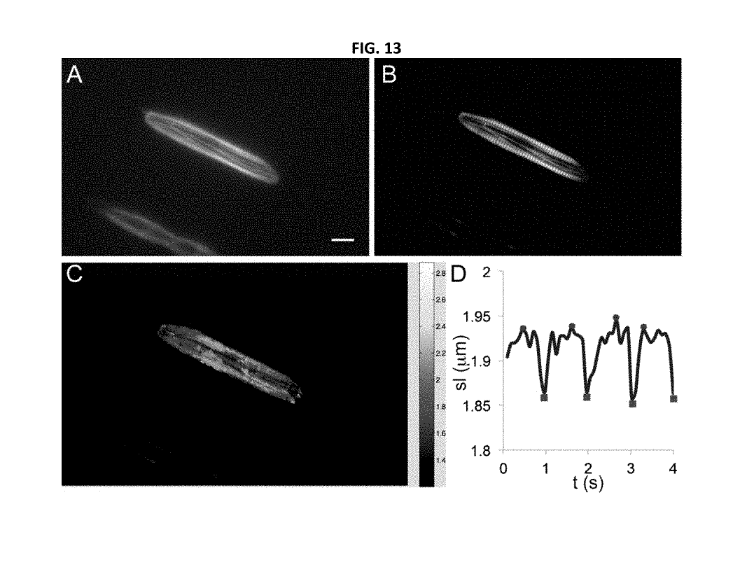

With this approach, we skeletonized sarcomeres for each frame (Online Movie XI and Online Movie XII), obtained heat maps of varying values of sl within single μpatterned hiPSC-CMs for each frame (Online Movie XIII) and calculated sarcomere shortening (ss) by subtraction the minimal values of average sl from the maximal values of sl that are calculated from contractile events captured in a video (FIG. 13). We then analyzed average sarcomere properties (FIG. 7) for the cell exposed to different concentrations of isoproterenol (FIG. 4 and Online Movies V, VI and VII) and for the cell where acute and chronic effects of omecamtiv mecarbil were captured in video (FIG. 6 and Online Movies VIII, IX and X). We calculated average sl values for all frames of the videos, maximal sl, minimal sl and ss (FIG. 7). With this analysis, we aimed to test if the variations in mechanical output that we observed for these cells related to changes in sl and ss and to test if measuring sarcomere properties can detect drug induced functional changes in μpatterned hiPSC-CMs. Both isoproterenol (FIG. 7A-D) and omecamtiv mecarbil (FIG. 7E-H) decreased average values of sl, but had different effects on ss. Isoproterenol-induced decrease in sl was accentuated with 1 μM (FIG. 7A), at which the maximal mean values of sl also decreased relative to what was observed in the cell before adding isoproterenol (FIG. 7B). Minimal average sl values decreased with 0.1 μM of isoproterenol and decrease even more at 1 μM. In addition, ss considerably increased with 0.1 μM, which may relate to the observed increase in mechanical output at this concentration (FIG. 4), but not with 1 μM. Chronic and acute effects of omecamtiv mecarbil also induced a decrease in average sl (FIG. 7E), in maximal average sl (FIG. 7F), in minimum average sl (FIG. 7G) and in average ss (FIG. 7H).

In summary we validated our approach for measuring sl within μpatterned hiPSC-CMs from videos of labeled myofibrils by also detecting drug-induced variations.

### D. Analyzing the Intracellular Asynchronicity of Movement Detects Defective Contractility

The intracellular space of a functional and mature primary CM beats synchronously during each contractile cycle. (Gulick 1991, Decker 1991, Forough 2011, Ibrahim 2011) Loss of synchronicity in muscular contractions is a marker of loss of function of cardiac muscle, which can originate from extracellular or intracellular disorders that lead to loss of heart function. (Tsai 2009, Roman-Campos 2013) Therefore, a more asynchronous contractile movement of the intracellular space should be an indicative of loss of function in beating μpatterned hiPSC-CMs. To test this hypothesis, we defined two parameters of asynchronicity (see Methods): spatial asynchronicity (a_{θ}) of contractile movement and temporal asynchronicity (a_{δ}) of contractile movement. a_{θ} was calculated from the direction of movement of all pixels in cells within videos. The parameter a_{θ} provides information on the amount of pixels that move along directions that are different from the average direction of movement with the ROI and on how different these directions are from the average. a_{δ} was calculated from the offset times (FIG. 8A) of each pixel within a region of interest (ROI) delimited by the borders of the cell (FIG. 8B) and provides information of when movement occurs in a cell region relative to the average timing of cell contractile movement (FIG. 8C). In a hiPSC-CM with no defective contractile function, all features within an ROI marking the cell borders should move more along the average direction of displacement and all pixels should simultaneously move. We tested the ability of the parameters a_{θ} and a_{δ} these to detect contractile defects with hiPSC-CMs.

For this purpose we assayed TALEN-engineered hiPSC-CMs with reduced expression of the MYBPC3 gene, which encodes for the myofilament protein myosin binding protein C. We analyzed the contractility of cells without one copy of MYBPC3 and without both copies of this gene (FIG. 8D-G). Low expression of MYBPCS3 had already been associated to pathological hypertrophy of the heart in mice, which involved disarray of the myocardium. (Harris 2002, Carrier 2004) We observed an increase in a_{θ} (FIG. 8D) and a_{δ} (FIG. 8E) in hiPSC-CMs with decreased expression of MYBPC3. In addition, these cells presented decreased values of \hat{t} and we also observed similar levels of decreased production of ΣF as previously reported by Birket and colleagues. (Birket 2015) These results validate the analysis of the asynchronicity of movement in μpatterned hiPSC-CMs to detect contractile defects that relate to loss of function.

### III. DISCUSSION

We present and validate an integrated approach to analyze the mechanical output of μpatterned hiPSC-CMs from microscopy videos acquired in a non-destructive manner (FIG. 1). From these analyses we obtain contractile and kinetic parameters that characterize the mechanical performance of μpatterned hiPSC-CMs, as well as information on sarcomere properties and intracellular synchronicity of movement. These approaches can detect the effects of drugs and mutations in cell contractility. Several methods have also already been developed by others to assay the mechanical output of single CMs, such as piezoelectric sensors, (Tribe 2007) atomic force microscopy (Domke 1999) or micropipette aspiration. (Sweitzer 1993) However, these techniques are more invasive, cell destructive and lower throughput than the presented platform. In addition, our approach does not require skilled technical expertise for acquiring and analyzing data. The integration of different video-based methods in the same computational platform facilitates the comparison of different parameters and increases the throughput of the presented level of functional analysis.

We mainly focused on testing the ability of the presented video-based analytical methods to quantify contractile changes in μpatterned hiPSC-CMs and detected alterations in the mechanical output of μpatterned hiPSC-CMs induced by caffeine, isoproterenol and omecamtiv mecarbil. Caffeine induced instantaneous contractile and kinetic changes right after being added to the cell culture medium (FIG. 3). Opening of calcium channels in the sarcoplasmic reticulum of CMs occurs upon adding caffeine leads to increased concentration of cytosolic calcium. (O'Neill 1990) We slowly increased the extracellular concentration of caffeine up to 10 μM to detect small changes in mechanical output. Sudden increase in the extracellular concentration of caffeine is known to instantaneously stop the beating of hiPSC-CMs due to depletion of calcium stores in the sarcoplasmic reticulum, which follows a fast increase in cytosolic calcium. (Itzhaki 2011) We observed a sudden increase in mechanical output right after adding caffeine, but also a sudden decrease in the kinetics of relaxation (FIG. 3C). The magnitude of the contractions that followed and the kinetics of relaxation considerably decreased as a consequence of increasing caffeine extracellular concentration (FIG. 3).

Isoproterenol is a beta-adrenergic agonist that affects a set of biological mechanisms that alter CM contractility (Wallukat 2002), but the specific contractile effects of isoproterenol also depend on its extracellular concentration. (Butler 2015, Katano 1992) The analysis of videos acquired when cells were exposed to different concentrations of isoproterenol (FIGS. 4 and 5) yielded results similar to what has been already reported in other studies to be the effects of isoproterenol. (Butler 2015, Yokoo 2009) The extracellular concentration of isoproterenol of 0.1 μM induced positive inotropic and positive chronotropic responses, while increasing the concentration of isoproterenol to 1 μM had a negative inotropic effect, but a stronger chronotropic response. In addition, our sarcomere length mapping approach showed that positive inotropic response related to increased sarcomere shortening (FIG. 7D). The same trend in the variation of contractile and kinetic parameters of mechanical output was obtained from analyzing the different videos (FIG. 4). However, one difference was observed in the magnitude of variation in mechanical output induced by 1 μM isoproterenol between the different analyses (FIG. 4). Specifically, traction force microscopy showed a dramatic decrease in ΣF and P (FIG. 4D,E) that was not identified from tracking with cross-correlation the displacement of myofibrils (FIG. 4F,G) or the cell displacement in brightfield (FIG. 4H,I). This difference suggests that variations in intracellular displacement may not directly relate to variations in force generation, even when presenting the same general trend. In addition, traction force microscopy (FIG. 5) performed better than cross-correlation of brightfield videos (FIG. 10) in detecting isoproterenol-induced variations in mechanical output from a population of imaged cells. However, previous studies have successfully used cross-correlation approaches to characterize mechanical output of hiPSC-CMs from brightfield videos. (Kijlstra 2015, Lan 2013, Huebsch 2016) Probably a higher number of analyzed cells would have revealed higher statistical significance between variations in parameters calculated from cross-correlation analysis (FIG. 10).

Analysis of cell movement (FIG. 1B-D) force estimation (FIG. 1E-I) and analysis of sarcomere movement (FIG. 1J-L) may not necessarily coincide because they result from videos of different imaged moving structures that are affected by different factors. Brightfield videos have information on the movement of the cell, which results from the movement of sarcomeres that is propagated through the intracellular environment. Therefore, the rheology of the sarcoplasmic milieu may affect the analyzed movement. The movement of microbeads in the substrate is a measure of how much a cell is pulling, which depends on the force generated by actin-myosin interactions and also on the intracellular balance of these forces and on the stability of extracellular adhesions. Imaging myofibrils in live cells may be the closest we get to analyze the basis of cell contractions: actin-myosin interactions. However, this method does not provide information on the number of phosphorylated myosin heads and on the number of active myosins. Therefore, cell movement, force generation and myofibril movement are related, but do not necessarily express the same cell contractile properties.

We also observed differences between the different types of outputs that result from analyzing the acute effects of omecamtiv mecarbil with traction force microscopy (FIG. 6D,E) and cross-correlation of brightfield videos (FIG. 6H,I). Omecamtiv mecarbil had unexpected effects in the contractility of μpatterned hiPSC-CMs (FIG. 10 and FIG. 6) and in chronically generating myofibril damages (FIG. 6C), while having different acute effects (FIG. 6D-I). The contractile and kinetic effects of omecamtiv mecarbil in CMs had already been shown to be atypical. (Butler 2015) Our results raise questions about potential mechanisms that may explain these effects of omecamtiv mecarbil, but require further investigation beyond the scope of this study. Omecamtiv mecarbil acts specifically on cardiac myosin by increasing the time of its strong actin-bound state (Liu 2016) and delays the relaxation of myofibrils. (Nagy 2015) In line with this information, our data show an increase in the time of contractions at higher concentrations of omecamtiv mecarbil (FIG. 10D) and increased rate of beating at lower concentrations of omecamtiv mecarbil (FIG. 10E). Chronic myofibril damages also require future study. Omecamtiv mecarbil leads to a significant shortening of sarcomeres (FIG. 7E-G). This change in sarcomere length suggests that the oscillatory contractions of sarcomeres and overlap between sarcomeres induced by omecamtiv mecarbil (FIG. 6B and Online Movie IX) may already result from an increase in intracellular tension. This suggestion is also supported by known relationships between calcium overload, tension and the contractile performance of sarcomeres. (ter Keurs, et al., 1980; Mulder et al., 1989; Davis et al., 2016; de Tombe et al., 2016)

Measuring asynchronicity of beating within μpatterned hiPSC-CMs was one of the novel methods that we developed to identify contractile defects in these cells. We tested the approaches for measuring asynchronicity with hiPSC-CMs expressing decreased levels of MYBPC3 (FIG. 8D,E). A decreased ability to generate contractile forces had also already been identified in hiPSC-CMs expressing low levels of MYBPC3. (Birket 2015) Our results validated the use of parameters of asynchronicity to detect contractile defects in μpatterned hiPSC-CMs. In summary, we have developed a multi-method platform to quantify different parameters that characterize the contractile activity of μpatterned hiPSC-CMs. Using three different types of videos allows a better understanding of contractile phenotypes taking into consideration how sarcomere properties and cell contractile movement relate to force generation. These combined capabilities can easily be applied for the study of mutations and of drug-induced contractile effects.

### IV. SUMMARY

### A. Rationale:

Cardiomyocytes generate the necessary mechanical output for heart function through contractile mechanisms that involve the shortening of sarcomeres along myofibrils. Human induced pluripotent stem cells can be differentiated into cardiomyocytes and better model the mechanical output of mature cardiomyocytes when micropatterned to assume physiological shapes. Quantifying the mechanical output of these cells evaluates the function of these cells and enables the ability of assaying cardiac activity in a dish.

### B. Objective:

Our goal was to develop and validate a computational platform that integrates analytical approaches to quantify the mechanical output of single micropatterned cardiomyocytes from videos acquired in a non-destructive and minimally invasive manner.

### C. Methods and Results:

We micropatterned single cardiomyocytes differentiated from human induced pluripotent stem cells on deformable polyacrylamide substrates containing fluorescent microbeads and labeled myofibrils. We then acquired videos of single beating cells, of the microbeads being displaced by contractile tractions and of moving myofibrils. These videos were independently analyzed to acquire parameters that characterize the mechanical output of single cells. We also developed novel methods to quantify sarcomere length from videos of moving myofibrils and to analyze loss of synchronicity of beating in cells with contractile defects. We tested this computational platform by detecting variations in mechanical output induced by drugs and in cells expressing low levels of myosin binding protein C. We observed that our method for analyzing contractile parameters may aid in better grasping the mechanisms that originate variations in the function of cardiomyocytes.

D. Conclusions:

We demonstrate that this computational platform can be used to assay cardiac function with cardiomyocytes differentiated from pluripotent stem cells. This tool can be further leveraged in future studies regarding the effects of mutations and drugs in cardiac function.

### IV. REFERENCES

Adrian R. J., Twenty years of particle image velocimetry, Experiments in Fluids 39, 159-169 (2005).
Birket M J, Ribeiro M C, Kosmidis G, Ward D, Leitoguinho A R, van de Pol V, Dambrot C, Devalla H D, Davis R P, Mastroberardino P G, Atsma D E, Passier R, Mummery C L. Contractile defect caused by mutation in mybpc3 revealed under conditions optimized for human psc-cardiomyocyte function. Cell reports. 2015; 13:733-745
Blaber J., Adair B. and Antoniou A., Ncorr: Open-Source 2D Digital Image Correlation Matlab Software, Experimental Mechanics 55, 1105-1122 (2015).
Brady A J. Mechanical properties of isolated cardiac myocytes. Physiological reviews. 1991; 71:413-428
Bray M. A., Sheehy S. P. and Parker K. K., Sarcomere alignment is regulated by myocyte shape, Cell Motil Cytoskeleton 65, 641-51 (2008).
Butler J. P., Tolic-Norrelykke I. M., Fabry B. and Fredberg J. J., Traction fields, moments, and strain energy that cells exert on their surroundings, Am J Physiol Cell Physiol 282, C595-605 (2002).
Butler L, Cros C, Oldman K L, Harmer A R, Pointon A, Pollard C E, Abi-Gerges N. Enhanced characterization of contractility in cardiomyocytes during early drug safety assessment. Toxicological sciences: an official journal of the Society of Toxicology. 2015; 145:396-406
Carrier L, Knoll R, Vignier N, Keller D I, Bausero P, Prudhon B, Isnard R, Ambroisine M L, Fiszman M, Ross J, Jr., Schwartz K, Chien K R. Asymmetric septal hypertrophy in heterozygous cmybp-c null mice. Cardiovascular research. 2004; 63:293-304
Davis J, Davis L C, Correll R N, Makarewich C A, Schwanekamp J A, Moussavi-Harami F, Wang D, York A J, Wu H, Houser S R, Seidman C E, Seidman J G, Regnier M, Metzger J M, Wu J C, Molkentin J D. A tension-based model distinguishes hypertrophic versus dilated cardiomyopathy. Cell. 2016; 165:1147-1159
de Tombe P P, ter Keurs H E. Cardiac muscle mechanics: Sarcomere length matters. Journal of molecular and cellular cardiology. 2016; 91:148-150
Decker M L, Behnke-Barclay M, Cook M G, Lesch M, Decker R S. Morphometric evaluation of the contractile apparatus in primary cultures of rabbit cardiac myocytes. Circulation research. 1991; 69:86-94
Dembo M., Oliver T., Ishihara A. and Jacobson K., Imaging the traction stresses exerted by locomoting cells with the elastic substratum method, Biophys J 70, 2008-22 (1996).
Domke J, Parak W J, George M, Gaub H E, Radmacher M. Mapping the mechanical pulse of single cardiomyocytes with the atomic force microscope. European biophysics journal: EBJ. 1999; 28:179-186
Forough R, Scarcello C, Perkins M. Cardiac biomarkers: A focus on cardiac regeneration. The journal of Tehran Heart Center. 2011; 6:179-186
Gulick T, Pieper S J, Murphy M A, Lange L G, Schreiner G F. A new method for assessment of cultured cardiac myocyte contractility detects immune factor-mediated inhibition of beta-adrenergic responses. Circulation. 1991; 84:313-321
Hansen P. C., Jensen T. K. and Rodriguez G., An adaptive pruning algorithm for the discrete L-curve criterion, Journal of Computational and Applied Mathematics 198, 483-492 (2007b).
Hansen P. C., Regularization Tools version 4.0 for Matlab 7.3, Numerical Algorithms 46, 189-194 (2007a).
Harris S P, Bartley C R, Hacker T A, McDonald K S, Douglas P S, Greaser M L, Powers P A, Moss R L. Hypertrophic cardiomyopathy in cardiac myosin binding protein-c knockout mice. Circulation research. 2002; 90:594-601
Huebsch N, Loskill P, Deveshwar N, Spencer C I, Judge L M, Mandegar M A, C B F, Mohamed T M, Ma Z, Mathur A, Sheehan A M, Truong A, Saxton M, Yoo J, Srivastava D, Desai T A, So P L, Healy K E, Conklin B R. Miniaturized ips-cell-derived cardiac muscles for physiologically relevant drug response analyses. Scientific reports. 2016; 6:24726
Ibrahim M, Gorelik J, Yacoub M H, Terracciano C M. The structure and function of cardiac t-tubules in health and disease. Proceedings of the Royal Society B: Biological Sciences. 2011; 278:2714-2723
Iribe G, Helmes M, Kohl P. Force-length relations in isolated intact cardiomyocytes subjected to dynamic changes in mechanical load. American journal of physiology. Heart and circulatory physiology. 2007; 292:H1487-1497
Itzhaki I, Rapoport S, Huber I, Mizrahi I, Zwi-Dantsis L, Arbel G, Schiller J, Gepstein L. Calcium handling in human induced pluripotent stem cell derived cardiomyocytes. PLoS ONE. 2011; 6
Kandow C. E., Georges P. C., Janmey P. A. and Beningo K. A., Polyacrylamide hydrogels for cell mechanics: steps toward optimization and alternative uses, Methods Cell Biol 83, 29-46 (2007).
Katano Y, Endoh M. Effects of a cardiotonic quinolinone derivative y-20487 on the isoproterenol-induced positive inotropic action and cyclic amp accumulation in rat ventricular myocardium: Comparison with rolipram, ro 20-1724, milrinone, and isobutylmethylxanthine. Journal of cardiovascular pharmacology. 1992; 20:715-722
Kijlstra J D, Hu D, Mittal N, Kausel E, van der Meer P, Garakani A, Domian I J. Integrated analysis of contractile kinetics, force generation, and electrical activity in single human stem cell-derived cardiomyocytes. Stem cell reports. 2015; 5:1226-1238
Kovesi P. D. 2000, “MATLAB and Octave Functions for Computer Vision and Image Processing,” ed.^{∧} eds. Editor.
Krause M., Hausherr J. M., Burgeth B., Herrmann C. and Krenkel W., Determination of the fibre orientation in composites using the structure tensor and local X-ray transform, Journal of Materials Science 45, 888-896 (2009).
Kuo P. L., Lee H., Bray M. A., Geisse N. A., Huang Y. T., Adams W. J., Sheehy S. P. and Parker K. K., Myocyte shape regulates lateral registry of sarcomeres and contractility, Am J Pathol 181, 2030-7 (2012).
Lan F, Lee A S, Liang P, Sanchez-Freire V, Nguyen P K, Wang L, Han L, Yen M, Wang Y, Sun N, Abilez O J, Hu S, Ebert A D, Navarrete E G, Simmons C S, Wheeler M, Pruitt B, Lewis R, Yamaguchi Y, Ashley E A, Bers D M, Robbins R C, Longaker M T, Wu J C. Abnormal calcium handling properties underlie familial hypertrophic cardiomyopathy pathology in patient-specific induced pluripotent stem cells. Cell stem cell. 2013; 12:101-113
Landau L. D., Lifshits E. M., Kosevich A. M. and Pitaevskiĭ L. P., Theory of elasticity. (Pergamon Press, Oxford Oxfordshire New York, 1986).
Lian X., Zhang J., Azarin S. M., Zhu K., Hazeltine L. B., Bao X., Hsiao C., Kamp T. J. and Palecek S. P., Directed cardiomyocyte differentiation from human pluripotent stem cells by modulating Wnt/β-catenin signaling under fully defined conditions, Nat Protoc 8, 162-75 (2013).
Lin H., Yifei W. and Jain A., Fingerprint image enhancement: algorithm and performance evaluation, IEEE Transactions on Pattern Analysis and Machine Intelligence 20, 777-789 (1998).
Linkert M., Rueden C. T., Allan C., Burel J. M., Moore W., Patterson A., Loranger B., Moore J., Neves C., Macdonald D., Tarkowska A., Sticco C., Hill E., Rossner M., Eliceiri K. W. and Swedlow J. R., Metadata matters: access to image data in the real world, J Cell Biol 189, 777-82 (2010).
Liu L C, Dorhout B, van der Meer P, Teerlink J R, Voors A A. Omecamtiv mecarbil: A new cardiac myosin activator for the treatment of heart failure. Expert opinion on investigational drugs. 2016; 25:117-127
McCormick N. and Lord J., Digital Image Correlation, Materials Today 13, 52-54 (2010).
Mulder B J, de Tombe P P, ter Keurs H E. Spontaneous and propagated contractions in rat cardiac trabeculae. The Journal of general physiology. 1989; 93:943-961
Munevar S., Wang Y. and Dembo M., Traction force microscopy of migrating normal and H-ras transformed 3T3 fibroblasts, Biophys J 80, 1744-57 (2001).
Nadal-Ginard B, Mandavi V. Molecular basis of cardiac performance. Plasticity of the myocardium generated through protein isoform switches. The Journal of clinical investigation. 1989; 84:1693-1700
Nagy L, Kovacs A, Bodi B, Pasztor E T, Fulop G A, Toth A, Edes I, Papp Z. The novel cardiac myosin activator omecamtiv mecarbil increases the calcium sensitivity of force production in isolated cardiomyocytes and skeletal muscle fibres of the rat. British journal of pharmacology. 2015
O'Neill S C, Eisner D A. A mechanism for the effects of caffeine on ca2+ release during diastole and systole in isolated rat ventricular myocytes. The Journal of physiology. 1990; 430:519-536
Ribeiro A J, Ang Y S, Fu J D, Rivas R N, Mohamed T M, Higgs G C, Srivastava D, Pruitt B L. Contractility of single cardiomyocytes differentiated from pluripotent stem cells depends on physiological shape and substrate stiffness. Proceedings of the National Academy of Sciences of the United States of America. 2015a; 112:12705-12710
Ribeiro A. J., Denisin A. K., Wilson R. E. and Pruitt B. L., For whom the cells pull: Hydrogel and micropost devices for measuring traction forces, Methods, (2015b).
Ribeiro M C, Tertoolen L G, Guadix J A, Bellin M, Kosmidis G, D′Aniello C, Monshouwer-Kloots J, Goumans M J, Wang Y L, Feinberg A W, Mummery C L, Passier R. Functional maturation of human pluripotent stem cell derived cardiomyocytes in vitro—correlation between contraction force and electrophysiology. Biomaterials. 2015c; 51:138-150
Ribeiro A. J., Zaleta-Rivera K., Ashley E. A. and Pruitt B. L., Stable, covalent attachment of laminin to microposts improves the contractility of mouse neonatal cardiomyocytes, ACS Appl Mater Interfaces 6, 15516-26 (2014).
Roman-Campos D, Sales-Junior P, Duarte H L, Gomes E R, Lara A, Campos P, Rocha N N, Resende R R, Ferreira A, Guatimosim S, Gazzinelli R T, Ropert C, Cruz J S. Novel insights into the development of chagasic cardiomyopathy: Role of pi3kinase/no axis. International journal of cardiology. 2013; 167:3011-3020
Sabass B., Gardel M. L., Waterman C. M. and Schwarz U. S., High Resolution Traction Force Microscopy Based on Experimental and Computational Advances, Biophys J 94, 207-20 (2008).
Schwarz U. S. and Soine J. R., Traction force microscopy on soft elastic substrates: A guide to recent computational advances, Biochim Biophys Acta 1853, 3095-104 (2015).
Schwarz U. S., Balaban N. Q., Riveline D., Bershadsky A., Geiger B. and Safran S. A., Calculation of forces at focal adhesions from elastic substrate data: the effect of localized force and the need for regularization, Biophys J 83, 1380-94 (2002).
Sweitzer N K, Moss R L. Determinants of loaded shortening velocity in single cardiac myocytes permeabilized with alpha-hemolysin. Circulation research. 1993; 73:1150-1162
Talkhabi M, Aghdami N, Baharvand H. Human cardiomyocyte generation from pluripotent stem cells: A state-of-art. Life sciences. 2016; 145:98-113
ter Keurs H E, Rijnsburger W H, van Heuningen R, Nagelsmit M J. Tension development and sarcomere length in rat cardiac trabeculae. Evidence of length-dependent activation. Circulation research. 1980; 46:703-714
Tohyama S., Hattori F., Sano M., Hishiki T., Nagahata Y., Matsuura T., Hashimoto H., Suzuki T., Yamashita H., Satoh Y., Egashira T., Seki T., Muraoka N., Yamakawa H., Ohgino Y., Tanaka T., Yoichi M., Yuasa S., Murata M., Suematsu M. and Fukuda K., Distinct metabolic flow enables large-scale purification of mouse and human pluripotent stem cell-derived cardiomyocytes, Cell Stem Cell 12, 127-37 (2013).
Tsai M S, Tang W, Sun S, Wang H, Freeman G, Chen W J, Weil M H. Individual effect of components of defibrillation waveform on the contractile function and intracellular calcium dynamics of cardiomyocytes. Critical care medicine. 2009; 37:2394-2401
Tseng Q., Duchemin-Pelletier E., Deshiere A., Balland M., Guillou H., Filhol O. and Thery M., Spatial organization of the extracellular matrix regulates cell-cell junction positioning, Proc Natl Acad Sci USA 109, 1506-11 (2012).
Wallukat G. The beta-adrenergic receptors. Herz. 2002; 27:683-690
Wang G., McCain M. L., Yang L., He A., Pasqualini F. S., Agarwal A., Yuan H., Jiang D., Zhang D., Zangi L., Geva J., Roberts A. E., Ma Q., Ding J., Chen J., Wang D. Z., Li K., Wang J., Wanders R. J., Kulik W., Vaz F. M., Laflamme M. A., Murry C. E., Chien K. R., Kelley R. I., Church G. M., Parker K. K. and Pu W. T., Modeling the mitochondrial cardiomyopathy of Barth syndrome with induced pluripotent stem cell and heart-on-chip technologies, Nat Med 20, 616-23 (2014).
Wen J. H., Vincent L. G., Fuhrmann A., Choi Y. S., Hribar K. C., Taylor-Weiner H., Chen S. and Engler A. J., Interplay of matrix stiffness and protein tethering in stem cell differentiation, Nat Mater 13, 979-87 (2014).
William T, Eize J S. Pivlab—time-resolved digital particle image velocimetry tool for matlab.
William T. and Eize J. S., PIVIab—Time-Resolved Digital Particle Image Velocimetry Tool for MATLAB.
Yang X, Pabon L, Murry C E. Engineering adolescence: Maturation of human pluripotent stem cell-derived cardiomyocytes. Circulation research. 2014; 114:511-523
Yokoo N, Baba S, Kaichi S, Niwa A, Mima T, Doi H, Yamanaka S, Nakahata T, Heike T. The effects of cardioactive drugs on cardiomyocytes derived from human induced pluripotent stem cells. Biochemical and biophysical research communications. 2009; 387:482-488

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A system for assaying human induced pluripotent stem cell-derived cardiomyocytes (hiPSC-CMs), the device comprising:
a traction force microscopy substrate (TFM substrate) having an adhesion protein domain on a surface thereof;
a video imager configured to obtain video data from an hiPSC-CM present on the adhesion protein domain; and
a processing module configured to receive the video data and derive a parameter of the hiPSC-CM therefrom.

2. The system according to claim 1, wherein the video data comprises bright field data.

3. The system according to claim 1, wherein the video data comprises fluorescence data.

4. The system according to claim 1, wherein the adhesion protein domain comprises one or more adhesion proteins.

5. The system according to claim 4, wherein the adhesion protein domain comprises a plurality of adhesion proteins.

6. The system according to claim 1, wherein the TFM substrate comprises a traction force microscopy hydrogel (TFM-hydrogel) and a surface of the TFM-hydrogel comprises two or more distinct adhesion protein domains.

7. The system according to claim 6, wherein a surface of the TFM-hydrogel comprises two or more distinct adhesion protein domains.

8. The system according to claim 1, wherein the TFM substrate comprises fluorescent microbeads.

9. The system according to claim 1, wherein the TFM substrate comprises crosslinks.

10. The system according to claim 1, wherein the parameter comprises a contractile dynamic parameter.

11. The system according to claim 1, wherein the parameter comprises a mechanical output parameter.

12. The system according to claim 1, wherein the parameter comprises a myofibril dynamic parameter.

13. The system according to claim 1, wherein the system comprises a positioner configured to place a hiPSC-CM on an adhesion protein domain.

14. The system according to claim 1, wherein the system comprises an introducer configured to selectively contact an active agent with an hiPSC-CM on an adhesion protein domain.

15. The system according to claim 1, where the system further comprises a retriever configured to remove a hiPSC-CM from the adhesion protein domain.

16. The system according to claim 15, wherein retriever is operably coupled to a cell analyzer.

17. A method for assaying human induced pluripotent stem cell-derived cardiomyocytes (hiPSC-CMs), the method comprising:
positioning a hiPSC-CM on an adhesion protein domain present on a surface of a traction force microscopy substrate (TFM substrate);
obtaining video data from the hiPSC-CM present on the adhesion protein domain; and
deriving a parameter of the hiPSC-CM from the obtained video data.

18-21. (canceled)

22. The method according to claim 17, wherein the TFM substrate comprises a traction force microscopy hydrogel (TFM-hydrogel).

23-28. (canceled)

29. The method according to claim 17, wherein the method further comprises selectively contacting an active agent with the hiPSC-CM on an adhesion protein domain.

30. The method according to claim 29, wherein the method comprises assessing the impact of the active agent on the hiPSC-CM.

31-32. (canceled)
